(19) United States
(12) Patent Application Publication
(10) Pub. No.: US 2011/0165676 A1
(43) Pub. Date: Jul. 7, 2011

(54) METHOD FOR DECELLULARIZATION

(75) Inventor: Richard A. Hopkins, Kansas City, MO (US)

(73) Assignee: The Children's Mercy Hospital, Kansas City, MO (US)

(21) Appl. No.: 12/813,487

(22) Filed: Jun. 10, 2010

Related U.S. Application Data
(60) Provisional application No. 61/258,666, filed on Nov. 6, 2009.

(51) Int. Cl. C12N 5/071; C12N 5/077

(52) U.S. Cl. 435/378; 435/325

(57) ABSTRACT

The present invention provides for decellularized tissue and method for decellularizing tissue. The method generally comprises the steps of obtaining a harvested tissue, performing a muscle shelf debridement, treating the tissue with an enzyme, washing the tissue with a detergent, and performing an organic solvent extraction on the tissue. The tissues decellularized according to the present invention have several advantages including removing more of the residual cell debris, dsDNA, and chemicals, as well as exhibiting less calcification and better ultimate tensile strength than tissues prepared not according to the method of the present invention.

### RELATED APPLICATIONS

This application relates to and claims priority to U.S. Provisional Patent Application No. 61/258,666, which was filed on Nov. 6, 2009, the contents and teachings of which are incorporated herein by reference.

### FIELD OF INVENTION

The field of invention relates to a treatment and method of treatment for tissues and tissue engineering as well as the product produced by such methods and treatments. Specifically, the invention relates to the field of tissue engineered constructs wherein the cells of the tissue are removed in preparation for engineering or cell seeding.

### BACKGROUND OF INVENTION

Numerous types of tissue engineered constructs and vascular grafts have been produced over the last few decades. Previous tissue constructs have included man-made polymers as substitutes for various portions of the organ to which the tissue belongs. Materials such as Teflon and Dacron have been used in various configurations such as scaffoldings, tissue engineered blood vessels, and the like. Nanofiber self-assemblies have been used as micro scaffolds upon which cells are grown. Textile technologies have been used in the preparation of non-woven meshes made of different polymers. The drawback to these types of technologies is that it is difficult to obtain adequate porosity and a regular pore size within the scaffold, having the effect of unsuccessful cell seeding. Solvent casting and particulate leaching is a technique that allows for an adequate pore size, but the thickness of the graft is limited. Another disadvantage of this technique is that organic solvents must be used and fully removed to avoid damage to cells seeded on the scaffold. This can be a long and difficult process. Gas foaming, where gas acts as a porogen, has been used to avoid the use of organic solvents. Gas foaming has the disadvantage of requiring unusually high temperatures in order to form the gas pores, prohibiting the incorporation of any temperature labile material into the polymer mix. Additionally, the pores do not form an interconnected structure. Emulsification or freeze-drying and thermally induced phase separation both have the disadvantage of irregular pore size and quality.

Tissue culturing using functional tissues and biological structures requires extensive culturing to promote survival, growth, and inducement of functionality. Very specific conditions, chemical treatment, temperature, and nutrients must be used to ensure a successful tissue graft. Most tissue culturing methods currently and previously used require an extensive time line for preparation of the tissue and functionality can be lost. These time intensive techniques produce tissue grafts in which new cells fail to maintain viability once introduced into the graft. Bioreactors have been used to maintain specific culture conditions as an improvement over previous methods. While Bioreactors help aid in consistent culture conditions, the specific pretreatment of the tissue along with other processes, such as pre and post cell seeding treatments, can make a dramatic difference in the quality of the final graft.

Tissue grafts of heart valve and other vascular components have been produced in the prior art. Cryopreserved homograft valves retain donor cells with varying degrees of metabolic activity. Cells become apoptotic as a result of harvest, transport, and cryopreservation rendering the valve essentially acellular nine to 12 months postimplantation. Retained antigenic, apoptotic, necrotic, donor cells and/or cellular debris lead to calcification and chronic inflammation, thereby promoting valve failure. An especially sensitive procedure is the decellularization process. The quality of the decellularization reflects on the quality of the graft as a whole and has an impact in the recellularization success of the graft.

Current protocols for decellularization have the disadvantage of leaving residual chemicals and other cell debris in the tissue, thus, hindering the new cells ability to grow on the biological scaffold. Another problem with current decellularization protocols is that the chemicals used to remove residual chemical and cell debris are so harsh that almost all extracellular matrix proteins are stripped from the graft. The loss of these extracellular matrix proteins impairs the ability of new cells to remain viable once seeded. Another problem with current tissue grafts is that they often become acellular 6-12 months after transplant due to issues with the graft's inability to retain viable cells. Additionally, the process of decellularization often leaves tissues soft, thereby making them more difficult to be handled, seeded, and transplanted. Accordingly, what is needed in the art is a protocol for decellularization which reduces the amount of residual chemical and cell debris, while being gentle enough to leave the extracellular matrix proteins intact. What is additionally needed is a method for decellularization that prevents the tissue from softening, thus providing a firm tissue graft ensuring future success of seeding, handling, and, ultimately, transplanting. Finally, what is needed is a decellularization process that promotes the retention of viable cells. What is additionally needed is a method for decellularization that results in acellular tissue suitable for freezing for long-term storage.

### SUMMARY OF INVENTION

The present invention overcomes the deficiencies of previous tissue grafts and provides distinct advantages over the prior art. Generally, the present invention provides methods, protocols, and solutions for preparing tissues for engineering applications by removing cells present in the tissue, namely, a decellularization process. A decellularized tissue prepared according to the method of the present invention is also provided by the present invention. Efficiently decellularized tissue engineered homografts, as provided for by the present invention, prolong durability of the homografts by reducing recipient inflammation, immune responses, fibrous scarring and calcification, ultimately decreasing the number of patients requiring multiple reconstructive cardiac surgeries.

The methods of the present invention generally comprise performing the following steps on a harvested tissue: a subvalvular muscle shelf debridement, an enzyme treatment, a detergent wash, and an organic solvent extraction. In one embodiment, the method generally comprises the steps of reciprocating osmotic shock sequences, a detergent wash, a RNA-DNA extraction, an enzyme treatment, and an organic solvent extraction. In a further embodiment, the method comprises the steps of a first reciprocating osmotic shock sequence, a first detergent wash, a second reciprocating osmotic shock sequence, a RNA-DNA extraction, an enzyme treatment, a second detergent wash, and an organic solvent extraction. In an additional embodiment, the method comprises a first reciprocating osmotic shock sequence, a detergent wash, a second reciprocating osmotic shock sequence, a RNA-DNA extraction, a digestion step, an enzyme treatment, a second detergent step, an organic solvent extraction, an ion-exchange detergent residual extraction, and a final organic extraction. In a particularly preferred embodiment, the method further comprises an additional washing step in addition to all of the steps noted above. This additional washing step is preferably performed after the second detergent step, but before the organic solvent extraction.

In another preferred embodiment, the method of the present invention comprises performing the following steps on a harvested tissue: a first wash in a Hypertonic Salt Solution, a first wash with Triton X®, a first rinse in ddH₂O, a second wash in a Hypertonic Salt Solution, a second rinse in ddH₂O, a second wash with Triton X®, a Benzonase® (EMD Chemicals—North American affiliate of Merck KGaA, Darmstadt, Germany) digest, a first wash in ddH₂O, a NLS (N-lauoylsarcosine Sodium Salt Solution) (1%) wash, a second ddH₂O wash, a Solvent Extraction in EtOH, an Organic Solvent Extraction, and an SMS (Saline-Mannitol Solution) wash or soak. This method is preferably carried out on heart valve and blood vessel tissue. In preferred forms, the first and second wash in the Hypertonic Salt Solution are performed for between 30 minutes and 4 hours, more preferably between about 1 hour and 3 hours, still more preferably between about 1½ hours and 2½ hours, and most preferably for about 2 hours. The first and second wash with Triton X® are performed for between about 1 to 5 hours, more preferably between about 1½ hours to 4½ hours, still more preferably between about 2 hours and 4 hours, even more preferably between about 2½ hours and 3½ hours, and most preferably for about 3 hours. The first ddH₂O rinse is preferably performed for between about 1 to 30 minutes, more preferably between about 2 to 25 minutes, still more preferably for between about 3 to 22 minutes, even more preferably for between about 4 and 20 minutes, more preferably between about 5 to 18 minutes, still more preferably for between about 6 to 16 minutes, even more preferably for between about 7 and 14 minutes, more preferably between about 8 to 12 minutes, still more preferably for between about 9 to 11 minutes, and most preferably for about 10 minutes. The second ddH₂O rinse is preferably performed for between about 10 to 180 minutes, more preferably between about 20 to 150 minutes, still more preferably for between about 30 to 120 minutes, even more preferably for between about 40 and 90 minutes, more preferably between about 50 to 75 minutes, still more preferably for between about 55 to 70 minutes, even more preferably for between about 57 to 65 minutes, more preferably between about 58 to 63 minutes, still more preferably for between about 59 to 61 minutes, and most preferably for about 60 minutes. Preferably, the Benzonase® digest is performed for about 6 to 18 hours, more preferably between about 7 to 17 hours, still more preferably between 8 to 16 hours, even more preferably between about 9 to 15 hours, more preferably between about 10 to 14 hours, even more preferably between about 11 to 13 hours, and most preferably for about 12 hours. Preferably, the first ddH₂O wash is performed for between about 5 minutes and 2 hours. Preferably, the NLS wash is performed for 2 to 48 hours, more preferably between about 4 to 44 hours, still more preferably between 8 to 40 hours, even more preferably between about 12 to 36 hours, more preferably between about 16 to 32 hours, even more preferably between about 20 to 28 hours, still more preferably between about 22 to 26 hours, and most preferably for about 24 hours. Preferably the NLS is between 0.5% and 2%, even more preferably between about 0.7% and 1.5%, and most preferably, the NLS is 1%. The second ddH₂O wash is performed for between about 10 minutes to 6 hours, more preferably between about 20 minutes to 5 hours, still more preferably between 30 minutes to 4½ hours, even more preferably between about 40 minutes to 4 hours, more preferably between about 1 hour to 3½ hours, even more preferably between about 1 hour and 20 minutes to 3 hours, still more preferably between about 1 hour and 40 minutes to 2½ hours, and most preferably for about 2 hours. The ddH₂O wash is preferably performed at about 5 RPM to 270 RPM, more preferably from about 10 RPM to 240 RPM, even more preferably between about 15 RPM and 200 RPM, still more preferably from about 25 RPM to 130 RPM, more preferably from about 30 RPM to 100 RPM, even more preferably from about 35 RPM to 70 RPM, still more preferably from about 40 RPM to 60 RPM, and most preferably at about 50 RPM. The solvent extraction is preferably performed with an alcohol, preferably EtOH, still more preferably 25% EtOH to 70% EtOH, even more preferably 30% EtOH to 50% EtOH, still more preferably 35% EtOH to 45% EtOH, and most preferably 40% EtOH. The shorter the amount of time the tissue is exposed to EtOH, the greater concentration of EtOH that can be used. Likewise, the greater amount of time the tissue is exposed to EtOH, the lesser the concentration of EtOH can be used. The concentration of EtOH should be high enough to perform the solvent extraction, but low enough so that the EtOH does not cause harm to the tissue. The solvent extraction is preferably performed for 10 to 75 minutes, more preferably from 15 to 65 minutes, even more preferably from 20 to 55 minutes, still more preferably from 25 to 40 minutes, and most preferably for about 30 minutes. The solvent extraction is preferably performed at about 5 RPM to 270 RPM, more preferably from about 10 RPM to 240 RPM, even more preferably between about 15 RPM and 200 RPM, still more preferably from about 25 RPM to 130 RPM, more preferably from about 30 RPM to 100 RPM, even more preferably from about 35 RPM to 70 RPM, still more preferably from about 40 RPM to 60 RPM, and most preferably at about 50 RPM. The solvent used for the solvent extraction is preferably EtOH. The Organic Solvent Extraction is generally performed for 2 to 48 hours, more preferably between about 4 to 44 hours, still more preferably between 8 to 40 hours, even more preferably between about 12 to 36 hours, more preferably between about 16 to 32 hours, even more preferably between about 20 to 28 hours, still more preferably between about 22 to 26 hours, and most preferably for about 24 hours. The SMS wash is preferably performed for between 30 minutes and 4 hours, more preferably between about 1 hour and 3 hours, still more preferably between about 1½ hours and 2½ hours, and most preferably for about 2 hours. The SMS wash is preferably performed at about 5 RPM to 270 RPM, more preferably from about 10 RPM to 240 RPM, even more preferably between about 15 RPM and 200 RPM, still more preferably from about 25 RPM to 130 RPM, more preferably from about 30 RPM to 100 RPM, even more preferably from about 35 RPM to 70 RPM, still more preferably from about 40 RPM to 60 RPM, and most preferably at about 50 RPM. The timing of these steps may be adjusted according to the type of tissue utilized. Further, the speed of the washes, soaks, and/or rinses in RPM will vary according to the type of rocker plate or shake incubator used as well as the type of motion created by the rocker plate or shake incubator. The speed of the rocker plate or shake incubator should be high enough to induce a mechanical agitation, but low enough to be gentle on the tissue such that the agitation does not harm the tissue.

For purposes of the present invention, a Hypertonic Salt Solution (HSS) and a Saline Mannitol Salt Solution (SMS) can be used interchangeably. Alternatively, any similar inorganic or organic composition or chemical that achieves similar osmolar strength to that of a HSS or SMS solution can be substituted for HSS or SMS for purposes of the present invention. Advantageously, these compositions dehydrate the tissue and prepare it for subsequent conditioning where the tissue is capable of more readily taking up or absorbing solutions in which the tissue is placed. In an embodiment where the tissue is going to be stored in solution for several days after the completion of the decellularization process, it is preferable to use a HSS solution before storing. Alternatively, in an embodiment where the tissue will be frozen, implanted, or recellularized shortly or immediately after the completion of the decellularization process, it is preferable to use a SMS solution before freezing, implanting, or recellularizing.

Preferably, all harvested tissues are harvested and stored according to the American Association of Tissue Banks Standards for Tissue Banking 12^{th} edition, the contents of which are herein incorporated by reference.

In a preferred embodiment the tissue is selected from mammalian tissue, avian tissue, or amphibian tissue. More preferably, the tissue is mammalian tissue, preferably selected from the group consisting of human, ovine, bovine, porcine, feline, canine, and combinations thereof. In a most preferred embodiment, the tissue is human tissue. The tissue to be decellularized can be any tissue suitable for use as a biological scaffold. Preferred tissues include, but are not limited to vascular tissue, organ tissue, digestive system tissue and muscle tissue, which include heart tissue, lung tissue, liver tissue, pancreas tissue, small and large intestine tissue, colon tissue, spleen tissue, gland tissue, and thyroid tissue, among others. In a most preferred embodiment, the tissue is vascular tissue, preferably heart valve tissue.

The timing of the method can be altered depending on the type of tissue, size of tissue, and other variables. Generally, the method takes about 2-14 days, but the appropriate amount of time can be determined by one of skill in the art. For example, in the case of a pulmonary valve, the method preferably takes about 2-7 days, more preferably, about 3-6 days, and, most preferably, about 3.5 to 4 days. In contrast, an aortic valve preferably takes about 3-9 days, more preferably, about 4-7 days, and, most preferably, about 5 days.

In one preferred embodiment, the reciprocating osmotic shock sequences include the use of a hypertonic salt solution. The sequence for the reciprocating osmotic shock sequences preferably includes treatment of tissue with a hypotonic solution, preferably double deionized water (“ddH₂O”), followed by a treatment of the tissue with a hypertonic salt solution, followed by a second treatment with a hypotonic solution, preferably ddH₂O. In some preferred forms or embodiments, the hypertonic salt solution includes one or more chlorides. In another preferred embodiment, the hypertonic salt solution comprises normal saline, one or more chlorides, a sugar or sugar alcohol, and combinations thereof. Preferred chlorides are selected from the group consisting of NaCl, MgCl₂, KCl, and combinations thereof. Still more preferably, the HSS solution comprising normal saline, one or more chlorides, and a sugar or sugar alcohol will further comprise NaCl in addition to the “one or more chlorides.” Various sugars or sugar alcohols including Mannitol, polysaccharides, polyolys, dulcitol, rhamitaol, inisitol, xylitol, sorbitol, rharrose, lactose, glucose, galactose, and combinations thereof are appropriate for use in the present invention. In a preferred embodiment, the sugar alcohol, preferably Mannitol, acts as a free-radical scavenger, removing harmful free radicals from the tissue to prevent damage. Any sugar or sugar alcohol having the properties of a free-radical scavenger are preferred for purposes of the present invention. In one preferred embodiment, the sugar alcohol is Mannitol. Preferably, the normal saline solution contains NaCl in an amount of about 0.2% to 5%, even more preferably from about 0.4%, to 4%, still more preferably from about 0.5% to about 3%, even more preferably from about 0.6% to about 2%, more preferably from about 0.7% to 1.5%, still more preferably from about 0.8% to about 1.2%, and most preferably about 0.9% by volume. Preferably, the chloride is present in the hypertonic salt solution in an amount of from about. When NaCl is present in the hypertonic salt solution, it is in an amount of from about 0.9% to 3.0% (w/v), more preferably, from about 1.2% to 2.7% (w/v), still more preferably, from about 1.5% to 2.3% (w/v), and most preferably, about 1.8% (w/v). When MgCl₂ is present in the hypertonic salt solution, it is in an amount of about 1.0 to 5.0 mM, more preferably, from about 1.5 to 4.0 mM, still more preferably, from about 2.0 to 3.0 mM, and most preferably, about 2.3 mM. When KCl is present in the hypertonic salt solution, it is generally in an amount of about 200 to 800 mM, more preferably, from about 300 to 700 mM, still more preferably, from about 400 to 600 mM, and most preferably about 500 mM. In a preferred embodiment, a sugar alcohol, preferably Mannitol, is present in the hypertonic salt solution in an amount of from about 5% to 20% (w/v), more preferably, from about 8% to 17% (w/v), still more preferably from about 10% to 15% (w/v), and most preferably about 12.5% (w/v) or about 683 mM. Preferably, the reciprocating osmotic shock sequences fracture the cell walls thereby allowing the enzyme and detergent washes to remove cellular debris.

In a preferred embodiment, the detergent wash includes the use of one or more detergents. The detergents can be nonionic, anionic, zwitterionic, detergents for the use of cell lysis, and combinations thereof. Any nonionic detergents can be used in the present invention. Preferred nonionic detergents include, but are not limited to: Chenodeoxycholic acid, Chenodeoxycholic acid sodium salt, Cholic acid, ox or sheep bile, Dehydrocholic acid, Deoxycholic acid, Deoxycholic acid methyl ester, Digitonin, Digitoxigenin, N,N-Dimethyldodecylamine N-oxide, Docusate sodium salt, Glycochenodeoxycholic acid sodium salt, Glycocholic acid hydrate, Glycocholic acid sodium salt hydrate, Glycocholic acid sodium salt, Glycolithocholic acid 3-sulfate disodium salt, Glycolithocholic acid ethyl ester, N-Laurolysarcosine sodium salt, N-Laurolysarcosine salt solution, Lithium dodecyl sulfate, Lugol solution, Niaproof 4, Triton®, Triton® QS-15, Triton® QS-44 solution, 1-Octanesulfonic acid sodium salt, Sodium 1-butanesulfonate, Sodium1-deccanesulfonate, Sodium1-dodecanesulfonate, Sodium 1-heptanesulfonate anhydrous, Sodium 1-nonanesulfonate, Sodium 1-propanesulfonate monohydrate, Sodium 2-bromoethanesulfonate, Sodium choleate hydrate, Sodium choleate, Sodium deoxycholate, Sodium deoxycholate monohydrate, Sodium dodecyl sulfate, Sodium hexanesulfonate anhydrous, Sodium octyl sulfate, Sodium pentanesulfonate anhydrous, Sodium taurocholate, Taurochenodeoxycholic acid sodium salt, Taurochenodeoxycholic acid sodium salt monohydrate, Taurochenodeoxycholic acid sodium salt hydrate, Taurolithocholic acid 3-sulfate disodium salt, Tauroursodeoxycholic acid sodium salt, Triton X®-200, Triton X®GS-20 solution, Trizma dodecyl sulfate, Ursodeoxycholic acid, and combinations thereof. Any anionic detergent will work for the purposes of the present invention. Preferred anionic detergents for use in the present invention, include, but are not limited to: BigCHAP, Bis (polyethylene glycol bis[imidazoyl carbonyl]), Brij®, Brij® 35, Brij® 56, Brij® 72, Brij® 76, Brij® 92V, Brij® 97, Brij® 58P, Cremophor® EL (Sigma, Aldrich), N-Decanoyl-N-methylglucamine, n-Decyl a-D-glucopyranoside, Decyl b-D-maltopyranoside, n-Dodecyl a-D-maltoside, Heptaethylene glycol monodecyl ether, n-Hexadecyl b-D-maltoside, Hexaethylene glycol monododecyl ether, Hexaethylene glycol monohexadecyl ether, Hexaethylene glycol monooctadecyl ether, Hexaethylene glycol monotetradecyl ether, Igepal CA-630, Methyl-6-O-(N-heptylcarbamoyl)-a-D-glucopyranoside, Nonaethylene glycol monododecyl ether, N-Nonanoyl-N-methylglucamine, Octaethylene glycol monodecyl ether, Octaethylene glycol monododecyl ether, Octaethylene glycolmonooctadecyl ether, Octaethylene glycol monotetradecyl ether, Octyl-b-D-glucopyranoside, Pentaethylene glycol monodecyl ether, Pentaethylene glycol monohexadecyl ether, Pentaethylene glycol monohexyl ether, Pentaethylene glycol monooctadecyl ether, Pentaethylene glycolmonooctyl ether, Polyethylene glycol ether, Polyoxyethylene, Saponin, Span® 20, Span® 40, Span® 60, Span® 65, Span® 80, Span® 85 (Sigma Aldrich), Tergitol, Tetradecyl-b-D-maltoside, Tetraethylene glycol monodecyl ether, Tetraethylene glycol monododecyl ether, Tetraethylene glycol monomonotetradecyl ether, Triton® CF-21, Triton® CF-32, Triton® DF-12, Triton® DF-16, Triton® GR-5M, Triton X®-100, Triton X®-102, Triton X®-15, Triton X®-151, Triton X®-207, Triton®, TWEEN® (Sigma Aldrich), Tyloxapol, n-Undecyl b-D-glucopyranoside, and combinations thereof. Any zwitterionic detergent will work for purposes of the present invention. Preferred zwitterionic detergents include, but are not limited to the following: CHAPS, CHAPSO, Sulfobetaine 3-10 (SB 3-10), Sulfobetaine 3-12 (SB 3-12), Sulfobetaine 3-14 (SB 3-14), ASB-14, ASB-16, ASB-C8Ø, Non-Detergent Sulfobetaine (ND SB) 201, DDMAB, DDMAU, EMPIGEN BB®Detergent, 30% Solution, Lauryldimethylamine Oxide (LDAO) 30% solution, ZWITTERGENT® 3-08 Detergent, ZWITTERGENT® 3-10 Detergent, ZWITTERGENT® 3-12 Detergent, ZWITTERGENT® 3-14 Detergent, ZWITTERGENT® 3-16 Detergent, and combinations thereof. In a particularly preferred embodiment, a nonionic detergent is used first followed by an anionic or zwitterionic detergent. In a preferred embodiment, the detergents used are Triton X®-100 (Triton), N-lauroylsarcosine Sodium Salt Solution (NLS), and combinations thereof. Preferably, the detergent wash has the effect of solubilizing proteins, lysing cells, and also acting as an anti-calcification agent. Generally, the detergent(s) is present in an amount of about 0.01% to 1% by volume, more preferably from about 0.02% to 0.7%, still more preferably from about 0.03% to 0.3%, even more preferably from about 0.04% to 0.09%, and most preferably about 0.05%.

In one preferred embodiment, the RNA-DNA extraction step comprises an enzyme. In another preferred embodiment, the RNA-DNA extraction comprises an enzyme, one or more salts, a base, and combinations thereof. Preferably the enzyme is a recombinant enzyme or endonuclease. Any endonuclease will work with the methods of the present invention. In a preferred embodiment, the enzyme is an endonuclease, even more preferably the endonuclease is Benzonase®. The endonuclease, preferably Benzonase®, is preferably present in the extraction in an amount of about 12.5 units, where one unit of Benzonase® is defined as the amount of enzyme that causes a ΔA₂₆₀ of 1.0 in 30 minutes, which corresponds to complete digestion of 37 μg of DNA (Novagen, United States). More preferably, Benzonase® is present in the extraction in an amount from about 0.01 to 0.5 KU/ml, more preferably from about 0.02 to 0.4 KU/ml, still more preferably from about 0.03 to 0.3 KU/ml, and most preferably about 0.0625 KU/ml. Preferably the endonuclease used has the property of removing DNA and RNA that is either single stranded, double stranded, linear or circular. Any endonuclease exhibiting similar properties is preferred for purposes of the present invention. Preferably the salt is a chloride, with one particularly preferred chloride being Magnesium chloride. In another preferred embodiment, the Benzonase® is present in a solution of MgCl₂. Preferably the MgCl₂ is present in an amount from about 2 to 15 mM solution of MgCl₂, more preferably from about 3 to 12 mM solution of MgCl₂, still more preferably from about 4 to 10 mM solution of MgCl₂, and is most preferably about an 8 mM solution of MgCl₂. The base is preferably a weak base, more preferably a hydroxide, and, even more preferably, ammonium hydroxide. In one preferred embodiment, the weak base, preferably ammonium hydroxide, is present in an amount from about 5 ul to about 40 ul, even more preferably from about 10 ul to about 30 ul, still more preferably from about 15 ul to about 22 ul, and is most preferably about 20 ul. Preferably, the RNA-DNA extraction has the effect of avoiding antigenicity issues and allowing for enzyme digestion.

In one preferred embodiment, the enzyme treatment step includes the use of a recombinant enzyme. The recombinant enzyme is preferably Benzonase®. Preferably, the enzyme treatment avoids antigenicity issues.

In another preferred embodiment, the organic solvent extraction step comprises an alcohol. The alcohol used can be any alcohol, and preferred alcohols are selected from, but are not limited to, the following group: ethyl alcohol, methyl alcohol, n-propyl alcohol, iso-propyl alcohol, n-butyl alcohol, sec-butyl alcohol, t-butyl alcohol, iso-amyl alcohol, n-decyl alcohol and combinations thereof. In one preferred embodiment, the alcohol has a high concentration, preferably from about 20 proof to 70 proof, even more preferably, from about 30 to 60 proof, still more preferably, from about 35 proof to 50 proof, and is most preferably about 40 proof. In preferred forms, the alcohol also acts an anti-calcification agent, one such preferred alcohol is ethyl alcohol. In another preferred embodiment, the organic solvent extraction step includes an ion-exchange detergent residual extraction. The ion-exchange detergent residual extraction preferably comprises microcarrier beads in an open reaction chamber where fluid is continually exchanged throughout the open reaction chamber. Preferably, the beads used in the ion-exchange detergent residual extraction are such that no residual beads are left on the tissue therefore minimizing bead-to-bead interaction. In one preferred embodiment, the extraction has the effect of sterilizing and disinfecting the valve, as well as removing lipids and other hydrophilic residuals. Preferably, the extraction step also has anti-calcification effects.

In a preferred embodiment, the organic extraction step comprises a salt. More preferably, the organic extraction comprises a salt, a saline solution, and water. Even more preferably, the organic extraction comprises a salt, a saline-sugar solution, and water. Preferably the salt is a chloride. In a preferred embodiment, the chloride is selected from the group consisting of NaCl, MgCl₂, KCl, and combinations thereof. Preferably the chloride is MgCl₂. In one preferred embodiment, the saline-sugar solution includes normal saline and a sugar alcohol. Preferably the sugar alcohol is selected from, but not limited to, the following: Glycol, Glycerol, Erythritol, Threitol, Arabitol, Cylitol, Ribitol, Sorbitol, Mannitol, Dulcitol, Iditol, Isomalt, Maltitol, and combinations thereof. Preferably, the sugar alcohol is Mannitol. Preferably, the organic extraction step has the effect of removing the extra water from the interstitium of the tissue reducing the “softening” effects and firming the tissue for safer handling and for better suturing, handling, and surgical characteristics.

In a preferred embodiment, the decellularization process of the present invention has the advantageous effect of preserving the extracellular matrix proteins within the tissue allowing the tissue to more easily and efficiently accept new cells, be surgically transplanted, and lead to a successful tissue in the recipient's body. Additionally, the decellularization process of the present invention has the effect of leaving the tissue relatively free from residual material left by the chemicals which contact the valve. This process allows for a cleaner, safer, and more efficient tissue.

In a preferred embodiment, the method further comprises additional washes or rinses throughout the method. Double-deionized water (ddH₂O) is preferably used to wash or rinse the valve. Preferably, there is at least one wash or rinse with ddH₂O, more preferably, there are at least two washes or rinses with ddH₂O, and most preferably, there are at least three washes or rinses with ddH₂O. The washes in ddH₂O can be performed in any container under any conditions suitable for the tissue being washed. In a preferred embodiment, the ddH₂O is placed in a container with the tissue and set on a rocker plate for the duration of the wash. Preferably, the rocker plate is used at a speed of 5 RPM to 270 RPM. The speed of the rocker plate depends on the movement of the rocker plate. Some rocker plates provide a shaking motion, a circular tilt, a belly dancer motion, or other type of motion. Each type of motion will require a different speed for the rocker plate. The appropriate rocker speed is one that provides a gentle mechanical agitation that does not disrupt the tissue to a point where it is harmful to the tissue.

Preferably, the decellularized tissues of the present invention provide adequate porosity and a regular pore size, such that these tissues are more easily recellularized. Since the tissues of the present invention are biological scaffolds, the pore size is biologically appropriate for the cells that will eventually recellularize the tissue, since the pores are the same size created by the body for the cells appropriate for the tissue.

The present invention has several benefits over other methods of decellularization. Preferably, the decellularized tissue of the present invention exhibits a considerable reduction in cellularity when compared to tissues decellularized using prior art methods, including cryopreserved tissue. Further, the method of the present invention produces decellularized tissue where very few or no cells are present in the tissue. Example 1 outlines a study in which it was determined heart valve tissue decellularized using methods in accordance with the present invention had a considerable reduction in cellularity, with no cells being present in the cusp and only spotty remnants of smooth muscle found in the arterial wall tissue. Additionally, tissue decellularized according to the method of the present invention is more easily recellularized and those cells remain in the tissue longer after implant, when compared to tissues prepared using prior art methods, including cryopreserved valves. Preferably, a decellularized tissue, prepared according to the methods of the present invention, is repopulated with cells by at least 10 weeks post-implant, more preferably, at least 15 weeks post-implant, more preferably, at least 20 weeks post-implant, and most preferably, at least 30 weeks post-implant. Additionally, tissues prepared according to the methods of the present invention are more easily repopulated by recipient autologous recellularization such that the seeded population is augmented by cell in-migration from surrounding tissues and circulation-based multipotential cells. It is additionally noted that tissues decellularized according to the methods of the present invention, that were recellularized, exhibited positive staining for desmin, vimentin, and αSMA, which is indicative a myofibroblast-like valve interstitial cell population. Example 1 illustrates that this staining was found in decellularized tissues and not in cryopreserved tissues or controls. Preferably, the decellularized tissue of the present invention also exhibits less calcification than found in bioprosthetic valves. Less calcification can be determined through several methods, including by analyzing pictures of the tissue. Other methods of determining calcification can be readily determined by those of skill in the art. It is also preferred that tissue decellularized according to the methods of the present invention has a reduced inflammatory response when compared to tissues not decellularized according to the methods of the present invention.

In a preferred embodiment, the decellularization method of the present invention removes double stranded DNA (“dsDNA”) from the tissue scaffold. As illustrated in Example 6, dsDNA was not detected in the decellularized heart valve in the leaflet or sinus regions in the valve. Preferably, after decellularization of tissue, according to the present invention, the amount of dsDNA found in the tissue is less than 0.0800 μg/mg, more preferably, less than 0.0500 μg/mg, still more preferably, less than 0.0100 μg/mg, more preferably, less than 0.0050 μg/mg, even more preferably less than 0.0010 μg/mg, more preferably, less than 0.0001 μg/mg, and, most preferably there is no dsDNA found in the tissue. Using the methods of the present invention, the dsDNA present in a tissue is reduced, when compared to those tissues not decellularized according to the present invention. This includes, but is not limited to, bioprosthetic valves and cryopreserved valves. It is additionally preferred that the amount of dsDNA in the tissue is determined by radiographic studies of the tissue.

In an additionally preferred embodiment, the peak temperature and enthalpy of tissue, when Differential Scanning Calorimetry (“DSC”) is analyzed, in tissue decellularized according to the present invention is higher than that of tissues decellularized using prior art methods or not decellularized according to the methods of the present invention. Example 7 illustrates that in decellularized heart valves, the peak temperature and enthalpy of the sinus and onset temperature and enthalpy of the wall were significantly higher than for those heart valves that were cryopreserved. Example 12 illustrates a second DSC investigation which provided for an equation for determining enthalpy. The equation for determining enthalpy is the area of the thermogram peak (j) over the mass of the dry tissue (g). Further, Example 12 provides for a p-value for differentiation of the onset temperature, peak temperature, and enthalpy of the leaftet, sinus, and wall of decellularized heart valves compared to cryopreserved heart valves.

Preferably, the decellularization process of the present invention results in an increase in the ultimate tensile strength (UTS), an increase in the elastic modulus of the tissue, and a decrease in the percentage of strain to failure of the tissue. Preferably, the increase or decrease is in comparison to a tissue that has not been decellularized according to the method of the present invention. Preferably, the UTS of the tissue is at least 100.00 kPa greater, more preferably, at least 171.3 kPa greater, even more preferably, at least 200 kPa greater, more preferably, at least 250 kPa greater, and, most preferably, at least 318.54 kPa greater than in those tissues not decellularized according to the present invention. Alternatively, the UTS of the tissue is at least 100.00 N/m greater, more preferably, at least 171.3 N/m greater, even more preferably, at least 200 N/m greater, more preferably, at least 250 N/m greater, and, most preferably, at least 343.02 N/m greater than in those tissues not decellularized according to the present invention. Preferably, the elastic modulus of the tissue is at least 300 kPa greater, more preferably, at least 400 kPa greater, still more preferably, at least 443.68 kPa greater, more preferably, at least 500 kPa greater, and, most preferably, at least 513.83 kPa greater than in those tissues not decellularized according to the present invention. Alternatively, the elastic modulus of the tissue is at least 500 N/m greater, more preferably, at least 600 N/m greater, still more preferably, at least 700 N/m greater, more preferably, at least 800 N/m greater, even more preferably at least 900 N/m greater, and most preferably, at least 911.53 N/m greater than in those tissues not decellularized according to the present invention. Preferably, when leaflet heart valve tissue is measured, it is measured in N/m. Preferably, when sinus wall and pulmonary artery tissue is measured, it is measured in kPa for both the UTS and the elastic modulus. As illustrated in Example 8, the decellularization of heart valve tissue according to the methods of the present invention resulted in a significant increase in the ultimate UTS of the leaflet and sinus tissue as well as a significant increase in the elastic modulus of the leaflet tissue.

Preferably, the decellularization process of the present invention reduces the antigenicity of the decellularized tissue. As illustrated in Example 9, the decellularization process of the present invention significantly reduces the level of MHC I expression in the valves tested for this study. Second, MHC I is a marker to predict the successful removal of cellular debris by the decellularization method of the present invention. In a preferred embodiment, the decellularized tissue of the present invention has a lower level of MHC I expression when compared to those tissues not decellularized according to the present invention. Example 13 provides for a second investigation to determine the level of MHC I expression in decellularized heart valves. Table 15 of Example 13 provides for the level of MHC I expression in decellularized heart valve portions compared to cryopreserved heart valve portions. It was surprisingly found that all of the decellularized heart valve portions had a low level of MHC I expression, compared to the cryopreserved valve portions that had either a normal or high level of MHC I expression.

An investigation was carried out to determine the differences between decellularized, cryopreserved, and bioprosthetic heart valves. Sheep were used as a model, divided into groups, and implanted with either decellularized heart valves, cryopreserved heart valves, or bioprosthetic heart valves. The decellularized heart valves were decellularized according to the methods of the present invention. The sheep were weighed and measured at 8, 20, 32, and 52 weeks post-surgery and an echocardiogram was used to evaluate valve function in the sheep. It was found that the bioprosthetic valves had a larger annulus diameter and wall thickness than the cryopreserved or decellularized valves. The bioprosthetic heart valves had a significantly smaller EOA, a higher peak, and mean than the cryopreserved or decellularized valves. The decellularized valves showed evidence of autologous recellularization in the conduit wall and extending into the base of cusp. Positive staining for desmin, vimentin, and α-SMA were found in explanted decellularized valves, which indicates a population of myofibroblast-like valve interstitial cells are present. The decellularized valves were found to perform at least as well as cryopreserved valves and are superior to xenograft valves and thus, are suitable to serve as a scaffold for the production of tissue engineered heart valves.

One embodiment of the present invention was carried out, as described in Example 2, where heart valves were decellularized over a 4 day period, according to the methods herein. On the first day of processing the detergent and osmotic shock sequences were performed, using HSS, Triton®. An RNA-DNA extraction was also performed on Day 1 using Benzonase. On day 2 of processing, the valves were rinsed for 1 hour in ddH₂O and then placed in NLS solution. An organic extraction was performed on day 3 using 40% EtOH, followed by an ion exchange detergent residual extraction was performed. On day 4, a Mannitol soak was performed. The valves decellularized according to this embodiment have better biomechanical properties, less calcification, little to no dsDNA and better elasticity than heart vavles prepared according to other methods, such as cryopreservation.

An alternate embodiment of the present invention was carried out, as described in Example 3. A 4 day decellularization process was carried out on harvested heart valves. On day 1 of the decellularizaiton process, the valves were rinsed in a Hypertonic Salt Solution (“HSS”), INLS, and ddH₂O followed by a wash in ddH₂O. The HSS solution contains NaCl, MgCl₂, and Mannitol. The valves were then placed in an overnight solution of Benzonase®, MgCl₂, and NH₄OH. On day 2, a detergent extraction was performed on the valves using a 1% NLS solution and ddH₂O. An organic solvent extraction was performed on day 3 using 25% EtOH, followed by an ion exchange detergent residual extraction using Bio-beads Dowex in a bioreactor. On day 4, the valves were washed in ddH₂O and then decanted in ddH₂O and PBS. Then, the valves were placed in a storage solution containing sheep plasma, X Amphotericin B, Pen/Strep, Levaquin, and Vancomcin. This embodiment of the decellularization process of the present invention has the result of removing cells and cell debris from the tissue without having a harmful effect on the valve. This preparation allows for donor cells to be easily transferred and grown in the decellularized tissue as well as preventing calcification to the heart valve.

A test for toxicity of the valves was completed as described in Example 4. The valves were harvested from sheep and then prepared in culture with a reagent. The valves were then incubated and the cultures were observed using a microscope to evaluate cellular characteristics and percent cell lysis. Additionally, the color of the media was observed, where a color shift towards a yellow media was associated with an acidic pH and a color shift towards magenta was associated with an alkaline pH. For the valves decellularized according to the methods of the present invention, the toxicity test showed no evidence of causing cell lysis of toxicity.

Yet another embodiment of the present invention was carried out as described in Example 5. This embodiment of the decellularizaiton process of the present invention took place over 5 days. On day 1, heart valves, which had been previously harvested, were subjected to a detergent and osmotic shock sequence. The valves were washed in HSS for 3 hours and then washed in Triton® for 3 hours. Each wash was conducted in a clean flask. The valves were then washed three times in ddH₂O for 15 minutes each wash. The valves were then washed a second time in HSS and then rinsed with ddH₂O. A second wash with Triton® was then performed and then the valves were transferred into flasks containing Benzonase®. The valves were then incubated in the Benzonase® overnight. Day 3 of the method included three rinses for 2 hours each in ddH₂O. On day 4, an organic solvent extraction was performed. First, the valves were washed in 40% EtOH. Then, an ion exchange detergent residual extraction was set up for each valve using EtOH and ddH₂O. The valves were removed from the extraction set up on Day 5 and rinsed in ddH₂O, normal saline, and then rinsed a second time in ddH₂O. The valves were then soaked in SMS for 1 hour. The result of this embodiment of the decellularization method of the present invention is that the valves have less cell debris and intact cells present within the tissue, without having a harmful effect on the valve. Less calcification was also observed in the valves when compared to valves not prepared according to the methods of the present invention.

Tissue, preferably, heart valves, contain little, if any double stranded DNA (dsDNA) within the scaffold after undergoing the decellularization process of the present invention. As illustrated in Example 6, valves were dissected into leaflet, sinus, and wall regions and the regions of the valves were prepared. The valves were either decellularized according to the methods of the present invention, cryopreserved, or were harvested and untreated controls. Next, dsDNA was isolated from the valve regions and analyzed using an HT Fluorometer. The amount of dsDNA per weight of the tissue was calculated for each of the decellularized, cryopreserved, and control valves. Results were reported for the average dsDNA concentration. Double Stranded DNA was detected in all cryopreserved pulmonary valve portions and this was consistent with the test control. In the decellularized valves dsDNA was not detected in the leaflet and sinus regions of the valve. A very low amount of dsDNA was detected in the wall region of the valve, but only in one out of the three dissected valves used in the investigation. The average dsDNA content in the cryopreserved valve portions was 0.0875±0.0257 μg/mg and the average dsDNA content for the test control was 0.1287±0.0083 μg/mg. The small amount of dsDNA detected in the wall portion of one of the three valves was 0.0001±0.0005 μg/mg.

Differential Scanning Calorimetry studies showed a significant difference in the peak temperature and enthalpy of the sinus and in the onset temperature and enthalpy of the wall in heart valves decellularized according to the present invention when compared to valves that were cryopreserved. The investigation leading to these findings is described in Example 7. DSC was carried out on decellularized and cryopreserved valves that were dissected into leaflet, sinus, and arterial wall portions. The valve portions were tested in the DSC by heating the tissue from 40° C. to 90° C. by 5° C. per minute to generate thermograms. Onset temperature, peak temperature, and enthalpy were collected and analyzed from the thermograms. The valves showed to be statistically significant between cryopreserved and decellularized valves for the peak temperature and enthalpy of the sinus and the onset temperature and enthalpy of the wall.

The biomechanical properties of tissue, preferably heart valves prepared according to the methods of the present invention, are better in decellularized tissue than tissue not decellularized according to the method of the present invention. This is illustrated in Example 8. Heart valves that were either decellularized according to the methods of the present or cryopreserved were used to determine the biomechanical properties. The investigation analyzed Ultimate Tensile Strength (UTS), and elastic modulus. Decellularization, according to the methods of the present invention, resulted in significant increases in the UTS of the leaflet and sinus tissue and the elastic modulus of the leaflet tissue when compared to the cryopreserved valves.

Advantageously, tissue decellularized according to the methods of the present invention were found to have less MHC I expression when compared to tissue not decellularized according to the methods of the present invention. This is illustrated in Example 9. Specifically, heart tissue was decellularized and analyzed for expression of MHC I using a Western Blot assay. It is a common problem with current valve replacement options that over time glutaraldehyde leaches out of the tissue components, which is indicative of the loss of collagen cross-linking. The loss of collagen cross-linking will eventually expose foreign antigens to the host, leading to calcification of the valve and ultimately, failure of the valve. It was found that the decellularization process of the present invention significantly reduces the level of MHC I expression. The investigation also determined that MHC I is a marker for predicting the successful removal of cellular debris. Thus, valves decellularized according to the method of the present invention contain less cellular debris, as this debris is removed successfully throughout the decellularization process.

A further embodiment of the decellularization method of the present invention is presented in Example 10. The investigation described in Example 10 took place over 4 days. This example provides a more detailed procedure for carrying out a further embodiment of the present invention. The heart valves decellularized according to this embodiment showed the removal of call debris from the valves without having a harmful effect. Less calcification, less dsDNA, and better mechanical properties were observed in the decellularized valves when compared to those valves that were not decellularized according to the methods of the present invention.

### DEFINITIONS

“Debridement”, as used herein, refers to processes by which dead, contaminated or adherent tissue or foreign materials are removed from a tissue. One type of debridement is an enzymatic debridement.

“Enzyme treatment”, as used herein, refers to the addition of an enzyme to a solution or treatment of a material, such as tissue, with an enzyme.

“Detergent Wash or rinse”, as used herein, refers to the washing, soaking, or rinsing of a tissue or solution with a detergent. The detergent can be any type of detergent including, but not limited to, nonionic detergents, anionic, zwitterionic, detergents for the use of cell lysis, and combinations thereof.

“Solvent Extraction”, as used herein, refers to the separation of materials of different chemical types and solubilities by selective solvent action. Some materials are separated more easily in one solvent than by another, hence there is a preferential extractive action. This process can be used to refine products, chemicals, etc.

“Osmotic Shock” as used herein, is a sudden change in the solute concentration around a cell causing rapid change in the movement of water across the cell membrane. This is possible under conditions of high concentrations of salts, substrates, or any solute in the supernatant causing water to be drawn out of the cells via osmosis. This process disrupts cell membranes and inhibits the transport of substrates and cofactors into the cell, thus, “shocking” and disrupting them, for easier removal of cells and cell debris.

“Organic Extraction” or “Organic Solvent Extraction”, for purposes of the present invention, refers to the “solvent extraction” described above, wherein said solvent is of organic nature.

“Digestion”, as used herein, refers to a chemical digestion. This also includes an enzymatic digestion.

“Decellularization”, for purposes of the present invention, refers to the process of removing cells and/or cellular debris from a tissue. In a preferred embodiment the decellularization process prepares tissue, such that it is available to accept new cells into its biological scaffold.

For purposes of the present invention, a “lower level” or “reduced” amount is in comparison to a tissue not decellularized according to the methods of the present invention. Preferably, the characteristic or property of the tissue decellularized in accordance with methods of the present invention is at least 10% lower or reduced by at least 10%. Conversely, a “higher level” or “increased” amount is in comparison to a tissue not decellularized according to the methods of the present invention. Preferably, the characteristic or property of the tissue decellularized in accordance with the methods of the present invention is at least 10% higher or increased by at least 10%. Tissues not decellularized according to the methods of the present invention include, but are not limited to, cryopreserved tissues, biomechanical tissues, and other types of scaffolds used for bioengineering or tissue engineering in the prior art.

Additionally, for the purposes of the present invention, all references to omega or Ω decell or decellularization process refer to the decell processes in accordance with the present invention.

In another preferred embodiment of the present invention, a method for removing cells from a tissue is provided. The method generally comprises the steps of obtaining a harvested tissue, performing a muscle shelf debridement on said tissue, treating said tissue with an enzyme, washing said tissue with a detergent, and performing an organic solvent extraction on said tissue. Preferably, the tissue is selected from the group consisting of: heart tissue, lung tissue, liver tissue, pancreas tissue, small intestine tissue, large intestine tissue, colon tissue, spleen tissue, and gland tissue. Of these, heart tissue, and especially an aortic or pulmonary heart valve, is particularly preferred. In some preferred forms, the method is completed over the course of 2-14 days, preferably over the course of 4-5 days. The detergent is preferably selected from the group consisting of a nonionic detergent, anionic detergent, zwitterionic detergent, and combinations thereof. In some preferred forms, a nonionic detergent is used first followed by the use of an anionic or zwitterionic detergent. Particularly preferred detergents are selected from the group consisting of Triton X®-100, N-lauroylsarcosine Sodium Salt Solution (NLS) and combinations thereof. When Triton X®-100 and/or said NLS are used, they are preferably present in an amount from about 0.04% to 0.6% by volume. One particularly preferred enzyme is Benzonase®. In preferred forms, the organic solvent extraction comprises the use of an alcohol, and the alcohol is preferably selected from the group consisting of ethyl alcohol, methyl alcohol, n-propyl alcohol, iso-propyl alcohol, n-butyl alcohol, sec-butyl alcohol, t-butyl alcohol, iso-amyl alcohol, n-decyl alcohol, and combinations thereof. Of these, ethyl alcohol is particularly preferred. In other preferred forms, the organic solvent extraction further comprises the use of a salt. Preferred salts are selected from the group consisting of NaCl, MgCl₂, KCL, and combinations thereof. In other preferred forms, the organic solvent extraction further comprises the use of a sugar alcohol with Mannitol being especially preferred. The muscle shelf debridement preferably comprises an enzymatic debridement by which dead, contaminated, or adherent tissue or foreign materials are removed from said tissue. Such methodology is effective at removing all, or essentially all of the dsDNA of the tissue, thereby reducing its antigenicity. Furthermore, such methodology results in a tissue especially adapted for recellularization and proliferation of autologous cells by a host receiving a transplant of the tissue.

In a further embodiment, the present invention provides a method for removing cells from a tissue generally comprising the steps of obtaining a harvested tissue, performing reciprocating osmotic shock sequences on said tissue, washing said tissue with a detergent, performing an RNA-DNA extraction on said tissue, treating said tissue with an enzyme, and performing an organic solvent extraction on said tissue. Preferably, the tissue is selected from the group consisting of: heart tissue, lung tissue, liver tissue, pancreas tissue, small intestine tissue, large intestine tissue, colon tissue, spleen tissue, and gland tissue, with heart tissue being especially preferred. When the tissue is heart tissue, it is preferably an aortic or pulmonary heart valve. Preferably, the method is completed over the course of 2-14 days, and more preferably, the method is completed over the course of 4 days. Preferably, the detergent is selected from the group consisting of a nonionic detergent, anionic detergent, zwitterionic detergent, and combinations thereof. In preferred forms, a nonionic detergent is used first followed by the use of an anionic or zwitterionic detergent. Preferred detergents are selected from the group consisting of Triton X®-100, N-lauroylsarcosine Sodium Salt Solution (NLS) and combinations thereof. When Triton X®-100 and/or said NLS are used as the detergent, they are preferably present in an amount from about 0.04% to 0.6% by volume. Preferably, the enzyme is an endonuclease with Benzonase® being particularly preferred. In preferred forms, the organic solvent extraction comprises the use of an alcohol. Preferably the alcohol is selected from the group consisting of ethyl alcohol, methyl alcohol, n-propyl alcohol, iso-propyl alcohol, n-butyl alcohol, sec-butyl alcohol, t-butyl alcohol, iso-amyl alcohol, n-decyl alcohol, and combinations thereof, with ethyl alcohol being particularly preferred. In some preferred forms, the organic solvent extraction further comprises the use of a salt. Preferred salts are selected from the group consisting of NaCl, MgCl₂, KCL, and combinations thereof. In some preferred forms, the organic solvent extraction further comprises the use of a sugar alcohol, with Mannitol being particularly preferred. Preferably, the osmotic shock sequences comprise the use of a Hypertonic Salt Solution. In preferred forms, the Hypertonic Salt Solution comprises one or more chlorides. Preferred chlorides are selected from the group consisting of NaCl, MgCl₂, KCL, and combinations thereof. In other preferred forms, the Hypertonic Salt Solution further comprises the use of a sugar alcohol, with Mannitol being particularly preferred. In some preferred forms, the RNA-DNA extraction comprises the use of an enzyme. The enzyme useful in methods of the present invention are preferably selected from the group consisting of a recombinant enzyme, an endonuclease, and combinations thereof, with an endonuclease being particularly preferred and Benzonase® being one such preferred endonuclease. In other preferred forms, the RNA-DNA extraction, even when using Benzonase® further includes the use of MgCl. In other preferred forms, the methods of the present invention further comprise the use of one or more washes of the tissue with ddH₂O.

In another embodiment of the present invention, a method for removing cells from a tissue is provided. The method generally comprises the steps of obtaining a harvested tissue, performing a first reciprocating osmotic shock sequence on said tissue, washing said tissue with a first detergent, performing a second reciprocating osmotic shock sequence on said tissue, performing a RNA-DNA extraction on said tissue, performing a digestion on said tissue, treating said tissue with an enzyme, washing said tissue with a second detergent, performing a first organic solvent extraction on said tissue, performing an ion-exchange detergent residual extraction on said tissue, and performing a second organic solvent extraction on said tissue. Variations of this method can be done according to the other methods described in more detail herein.

In another embodiment of the present invention, a method for removing the cells from a tissue is provided. The method generally comprises the steps of a first washing of said tissue in a Hypertonic Salt Solution, a first washing of said tissue in Triton X®, a first rinsing of said tissue in ddH₂O, a second washing of said tissue in a Hypertonic Salt Solution, a second rinsing of said tissue in ddH₂O, a second washing of said tissue in Triton X®, performing a Benzonase® digestion on said tissue, a third rinsing of said tissue in ddH₂O, a washing of the tissue in a 1% NLS solution, a fourth rinsing of said tissue in ddH₂O, washing said tissue with 40% EtOH, performing an organic solvent extraction on said tissue, and washing said tissue with SMS. Variations of this method can be done according to the other methods described in more detail herein.

In another embodiment of the present invention, a decellularized tissue is provided. This decellularized tissue can be prepared using any method described herein. Preferably, after the decellularization process, the tissue will contain little or no dsDNA. Advantageously, tissues decellularized using methods of the present invention exhibit less calcification after implant when compared to tissues prepared not according to the methods of the present invention. Another characteristic of a tissue decellularized in accordance with the present invention is that the tissue has a reduced inflammatory response when compared to tissues prepared not according to method of the present invention. Advantageously, tissues decellularized in accordance with methods of the present invention have a higher ultimate tensile strength and elastic modulus when compared to tissues not prepared according to the methods of the present invention.

### BRIEF DESCRIPTION OF THE FIGURES

FIG. 1 illustrates the body surface area (m²) calculated as 0.09*BW(kg)^{0.67} of cryopreserved, bioprosthetic, and decellularized heart valves at 0, 8, 20, 32, and 52 weeks post implant;

FIG. 2 illustrates the physical characteristics of implanted valves measured at the time of implant into the right ventricular outflow track of juvenile sheep;

FIG. 3 illustrates a gross photograph (3A) and radiograph (3B) of a decellularized valve at explant (20 weeks). No cusp or wall calcification was observed;

FIG. 4 illustrates a gross photograph (4A) and radiograph (4B) of a cryopreserved vale at explant (20 weeks). Calcification of the arterial wall and suture lines were observed;

FIG. 5 illustrates a gross photograph (5A) and radiograph (5B) of a xenograft valve at explant (20 weeks). Calcification of the cusp, arterial wall, and suture lines were observed;

FIG. 6 illustrates a decellularized (6A) and native (6B) pulmonary valve cusps. The decellularized cusps were rendered free of cells and cell debris;

FIG. 7 illustrates decellularized valves at explant where the decellularized valves remained acellular at the midpoint and end of the cusps (7A). In vivo recellularization was observed radiating from the base of the cusps and extending into the spongiosa (7B);

FIG. 8 illustrates cryopreserved valves at explant that showed reduced cellularity throughout the base (8A) and mid-portion (8B) of the valve cusp;

FIG. 9 illustrates xenograft valves at explant where the xenograft valves retained cellularity (9B) but showed wall calcification (9B);

FIG. 10 illustrates the mean onset temperature of a decellularized valve versus a cryopreserved valve;

FIG. 11 illustrates the mean peak temperature of a decellularized valve versus a cryopreserved valve;

FIG. 12 illustrates the mean enthalpy of a decellularized valve versus a cryopreserved valve;

FIG. 13 illustrates MHC I expression in decellularized valves versus cryopreserved valves, where there is a drastic difference in detection of MHC I in decellularized valves when compared to cryopreserved valves;

FIG. 14 illustrates MHC I expression in decellularized valves when compared to the positive control; and

FIG. 15 illustrates the bioreactor set up for the organic extraction; and

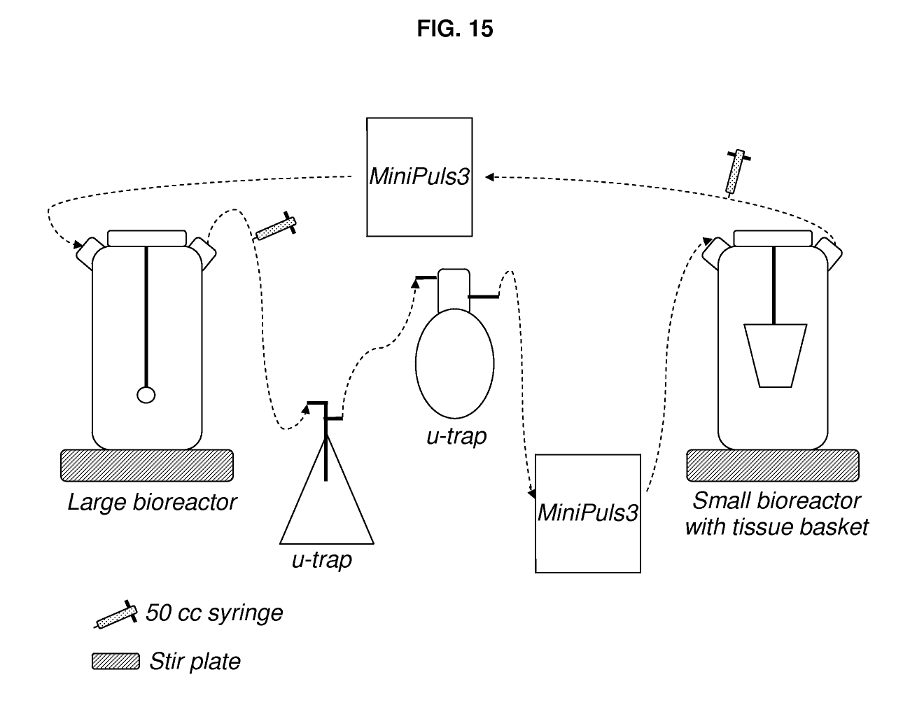

FIG. 16 illustrates the three cusps that the cryopreserved valves were dissected into in Example 12 and 13.

### DETAILED DESCRIPTION OF THE PREFERRED EMBODIMENTS

The following examples are representative of preferred embodiments of the present invention. It is understood that nothing herein should be taken as a limitation upon the overall invention.

### Example 1

This example illustrates analysis of the differences between decellularized, cryopreserved, and bioprosthetic heart valves.

### Materials and Methods

### Animals

All animal procedures were carried out under protocols approved by the Institutional Animal Care and Use Committee and animals received humane care in compliance with the Guide for Care and Use of Laboratory Animals (NIH Publication #85-23). Nineteen female domestic sheep (ovis ares; Suffolk/North Country Cheviot; 160±9d, 46.5±9 kg) were divided into three treatment groups. Group 1 sheep (n=8) were implanted with cryopreserved homografts further treated with a series of steps resulting in the decellularization of the tissue. Group 2 sheep (n=6) were implanted with cryopreserved homografts and Group 3 sheep (n=4) were implanted with a commercially available glutaraldehyde-preserved porcine aortic root bioprostheses (Freestyle, Medtronic, Minneapolis, Minn.). Sheep were survived for either 20 wk (Group 1, n=4; Group 2, n=3; Group 3, n=2) or 52 wk (Group 1, n=4; Group 2, n=3; Group 3, n=2).

### Homograft Harvest and Processing

Male domestic sheep (Suffolk/North Country Cheviot; 176±48d; 46±5 kg) were selected from non-related herds to serve as donor animals. Briefly, donor animals were euthanized with sodium pentobarbital and prepared for sterile heart harvest. Warm ischemic time was less than one hour. Following removal, the heart was rinsed in 500 mL sterile Lactated Ringer's solution. Pulmonary valves were dissected free from the heart, rinsed in 200 mL sterile Lactated Ringer's solution and placed in an antibiotic solution containing 4% amphotericin-B (Sigma-Aldrich, St. Louis, Mo.), 4% penicillin-streptomycin (Sigma-Aldrich) in Lactated Ringer's solution (92%, Baxter, Deerfield, Ill.). Valves were stored at 4° C. until further processed, with a cold ischemic time of 72 hours.

Group 2 valves were cryopreserved with clinically analogous protocols using 10% dimethylsulfoxide and 10% fetal bovine serum in RPMI-1640 (Gibco, Carlsbad, Calif.). Valves were frozen at −1° C./minute using a computer-controlled freezing system (Custom Biogenic Systems, Romeo, Mich.) and stored at ≦160° C. for at least 48 hours prior to thawing and implantation. Valves were thawed using clinically analogous protocols wherein each valve was held at room temperature for 7 minutes followed by a 7 minute bath of warm (37° C.) sterile normal saline. Valves were placed in a final sterile normal saline bath until implantation.

### Decellularization

A novel (previously unpublished) decellularization technique was used in this study. Briefly, following the completion of the 72 hours cold ischemic time, Group 1 valves were decellularized using a series of reagents including two anionic, non-denaturing detergents (n-lauroyl sarcosine, Triton-X®; Sigma-Aldrich), reciprocating osmolality wash solutions and recombinant endonuclease (Benzonase®, EMD Biosciences, Gibbstown, N.J.). Following these steps, valves were rinsed for 24 hours at room temperature in sterile deionized water recirculated through a bed of ion exchange resins (Amberlite XAD, Dowex Monosphere, IWT-TMD-8; Sigma-Aldrich). Following the completion of decellularization, valves were stored at 4° C. in a Lactated Ringer's-based solution containing, amphotericin-B (4%), penicillin-streptomycin (2%), vancomycin (25 μg/mL; Sigma-Aldrich), mannitol (25 μg/mL) and ciprofloxacin (40 μg/mL) until implantation.

### Surgical Methods

On the day of surgery, animals were anesthetized with intravenous propofol (4-6 mg/kg) followed by intubation and administration of isoflurane anesthesia (0.5-5%). A left thoracotomy was performed and the animal placed on cardiopulmonary bypass. Following removal of the native pulmonary valve leaflets, the replacement valve was sewn in using 4-0 (proximal anastomosis) and 5-0 (distal anastomosis) running Prolene suture (Ethicon, Cornelia, Ga.). Cryopreserved homograft valves were prepared as previously described. Decellularized homograft valves and cryopreserved valves were rinsed in sterile normal saline prior to implantation. Aortic root bioprostheses were removed from the packaging material and rinsed three times in sterile normal saline for 5 minutes each rinse, for a total of 15 minutes of rinsing prior to implantation.

Immediately preceding implantation, each valve was measured using calipers and a ruler to determine the size of the annulus and sino-tubular junction, as well as the thickness of the pulmonary artery or aorta. The native pulmonary artery was also measured at the division site. Following implantation, the diameter of the test valve was measured at the proximal and distal anastomoses as well as at the midpoint. The total graft length was also measured.

To document decellularization as compared to the control grafts, a small section of each graft wall was taken prior to implant, rinsed in normal saline and placed into HistoChoice MB (Amresco, Inc., Solon, Ohio) and stored at 4° C. until histological analysis.

Following implant, the animals were weaned off cardiopulmonary bypass and the chest closed. Post-operatively, the animals were given buprenorphine (0.005-0.01 mg/kg) and fentanyl (3-5 ug/kg) for pain management.

### Serial Studies

### Animal Growth

Animals were weighed using a livestock scale and measured (nucchal ridge to base of tail) on the day of surgery and at 8, 20, 32 and 52 weeks post-surgery. Body surface area (“BSA”) was calculated using the Haycock formula for transesophageal echocardiography data analysis:

BSA(m²)=0.024265×wt(kg)^{0.5378}×height(cm)^{0.3964}

Sheep body surface area was also calculated using the formula of Mitchell (1928)

0.09*BW(kg)^{0.67}.

### Echocardiography

Transesophageal echocardiography (TEE) was used to evaluate valve function in recipient animals following pulmonary valve replacement and to screen donor animals for functional pulmonary valves prior to harvest. All donor animals were found to have satisfactory valve function prior to harvest. In recipient animals, TEE was carried out immediately following surgery and at 8±2, 20±2, 32±2 and 52±2 weeks following surgery. A TEE probe (X7-2T; Philips, Amsterdam, The Netherlands) was inserted through a protective bite block into the esophagus and two dimensional images and Doppler-derived hemodynamic measurements were obtained of the pulmonary valve allograft and other relevant cardiac structures. Images and measurements were digitally captured and archived on an ultrasound platform (iE33; Philips). During each exam, the internal diameter of the right ventricular outflow tract (RVOT) and the graft at the annulus were recorded, as was a description of leaflet excursion and indications of the presence or absence of vegetations and calcifications. Doppler-derived blood flow velocities across the RVOT and across the implanted pulmonary valve were obtained and used to calculate mean and peak pressure gradients via the modified Bernoulli equation (ΔP=4V²) and cardiac output. The effective orifice area was calculated via a modified continuity equation, which was further normalized to BSA (EOA index; EOAI). Color flow Doppler was used to evaluate valvular regurgitation, assessed as none (no regurgitant jet), trace (regurgitant jet limited to immediate valve area), mild (regurgitant jet limited to RVOT), moderate (regurgitant jet extending into the right ventricular cavity) or severe (regurgitant jet extended to the tricuspid valve).

### Pre-Explant Studies

### Cardiac Catheterization

Cardiac catheterization occurred on the day of termination (20±2 or 52±2 weeks post-implant). Following the induction of general anesthesia (propofol, 4-6 mg/kg IV), the right femoral artery and vein were exposed. A 7-French introducer sheath was placed into the right femoral vein. A 7-French Bermann angiographic catheter was placed into the sheath, threaded up the inferior vena cava and right atrium and placed into the superior vena cava (SVC). While in the SVC, oxygen saturation was obtained using a small sample of blood. The catheter was then withdrawn into the right atrium and atrial a-wave, v-wave and mean pressure obtained. The catheter was then advanced into the right ventricle (RV) and systolic pressure, end-diastolic pressure and RV oxygen saturation obtained. The pulmonary artery and valve was imaged using angiography and two views (45 degrees cranial, 0 degrees LAO; 0 degrees cranial, 90 degrees LAO) with an object of known size used for calibration in both views. A power injection of 75 mL contrast at 25 mL per second (1 second rise) was made below the valve to assess stenosis, leaflet motion, ventricular function (where applicable). Data included valve diameters at the right ventricular outflow tract, sinuses, distal main pulmonary artery, proximal anastomosis, distal anastomosis, sino-tubular junction and valve annulus; right atrial a-wave, v-wave and mean pressures; right ventricular systolic and diastolic pressures; superior vena cava and right ventricular saturations; stenosis (normal leaflet thickness and mobility), mild (mildly increased leaflet thickness with mildly decreased mobility), moderate (moderately increased leaflet thickness with clearly diminished mobility), or severe (markedly increased leaflet thickness with significantly reduced mobility); and regurgitation (no reflux of contrast into ventricle), mild (minimal contrast reflux into ventricle, clears with every beat), moderate (contrast easily seen to reflux into ventricle, clears after 2-3 beats), or severe (significant opacity of ventricle with contrast, clears after prolonged time).

### Valve Explant Studies

### Gross Examination

Animals surviving to 20 or 52 weeks were euthanized with an overdose of sodium pentobarbital and the entire heart-lung block excised. Animals were subjected to necropsy and tissue samples of the spleen, kidney, liver, lungs and ventricles were placed in 10% neutral buffered formalin. Animals that did not survive until scheduled explant (found dead) were necropsied in the same fashion. Following dissection of the implanted valve, gross observations were recorded on a standardized valve diagram. The circumference of the valve was measured using calipers or Hegar dilators at the distal and proximal anastomoses and the midpoint of the graft. The length of the graft was also measured. Following measurement, the valve was rinsed in Lactated Ringer's solution and photographed. Fresh explants were also examined under a dissecting microscope (Stereodiscovery v12, Karl Zeiss, Thornwood, N.Y.), photographed (Sony CyberShot, San Diego, Calif.) and radiographed (Faxitron-LR, Lincolnshire, Ill.).

### Histopathology

Valve conduit sections taken at implant were embedded in paraffin and Hematoxylin and Eosin (H&E) stains were prepared by a commercial histology laboratory (American Histo Labs, Gaithersburg, Md.).

Following gross observations at explant, valves were dissected longitudinally along the cuspal commissures and placed into preservative (Histochoice-MB). For histological evaluation, one-third of each cusp was further dissected through the valve, extending from the base to the free edge of the leaflet and also including the pulmonary artery and sub-valvular muscle above and below the anastamoses. The specimens were embedded in paraffin and Movat pentachrome stains, H&E stains and unstained sections were prepared by a commercial histology laboratory. Immunohistochemistry was carried out on unstained sections following deparaffinization in xylenes, rehydration through sequential alcohol immersion and antigen retrieval via incubation in a citrate-based antigen retrieval solution (Vector Labs, Burlingame, Calif.) for 10 minutes at 90° C. Primary antibodies included α-smooth muscle actin (α-SMA; mouse monoclonal; Dako, Carpinteria, Calif.), desmin (rabbit polyclonal; Neomarkers, Fremont, Calif.), vimentin (rabbit polyclonal; Neomarkers), and factor VIII (rabbit polyclonal; Dako) were incubated overnight at 4° C. followed by exposure to either an alkaline phosphatase or fluorochrome labeled secondary antibody. Samples to which no primary antibody was added (buffer only) were used as negative tissue controls.

Slides were reviewed using a light microscope equipped with digital camera and software (Axioimager Z1; Axiovision; Karl Zeiss), followed by further processing by Photoshop Elements (Adobe Systems, San Jose, Calif., USA).

### Statistical Analysis

All statistical analyses were performed using the SPSS statistical software package (v. 17, Chicago, Ill.). Serially-measured continuous variables were analyzed by mixed-models repeated measures analysis of variance (ANOVA). Fixed model effects included time, treatment and the treatment by time interaction and the random effect was subject. Variables measured at only a single point during the study were analyzed using a general linear models ANOVA. For all ANOVA analyses, the appropriate correlation matrix was chosen based on the smallest Akaike's Information Criteria and post-hoc mean comparisons were made using Bonferroni multiple significance tests. Categorical variables were evaluated using the Pearson chi squared analysis and are presented as median±range. Data are presented as mean±standard error (continuous variables) or median±range (categorical variables) and statistical significance was set at P<0.05.

### Results and Conclusions

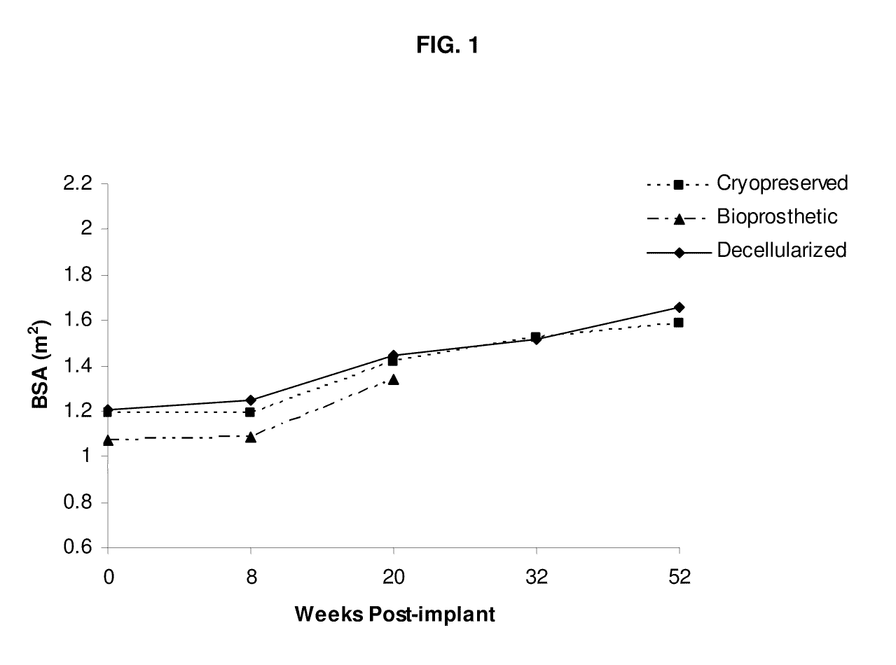

Of the 18 animals enrolled in the study, three died prematurely (prior to scheduled explant) of endocarditis (n=2, bioprosthetic; n=1, cryopreserved) and were excluded from the analysis. One additional animal in the decellularized group was also excluded from the analysis due to the presence of a calcified nodule at explant, suggestive of healed endocarditis. Animal growth, measured as BSA, increased similarly in all treatment groups (P=0.45) over the course of the study (P=0.001; FIG. 1).

Implant Characteristics

All valves were implanted successfully during routine left thoracotomy and cardiopulmonary bypass. All three valve types had identical scores of 5 (range, 1=poor to 5=excellent) for kink resistance, visualization of the surgical field and ease of control of suture line bleeding (X², P<0.05). However, the decellularized and cryopreserved valves scored better than the bioprosthetic valves for all other surgical characteristics (Table 1).

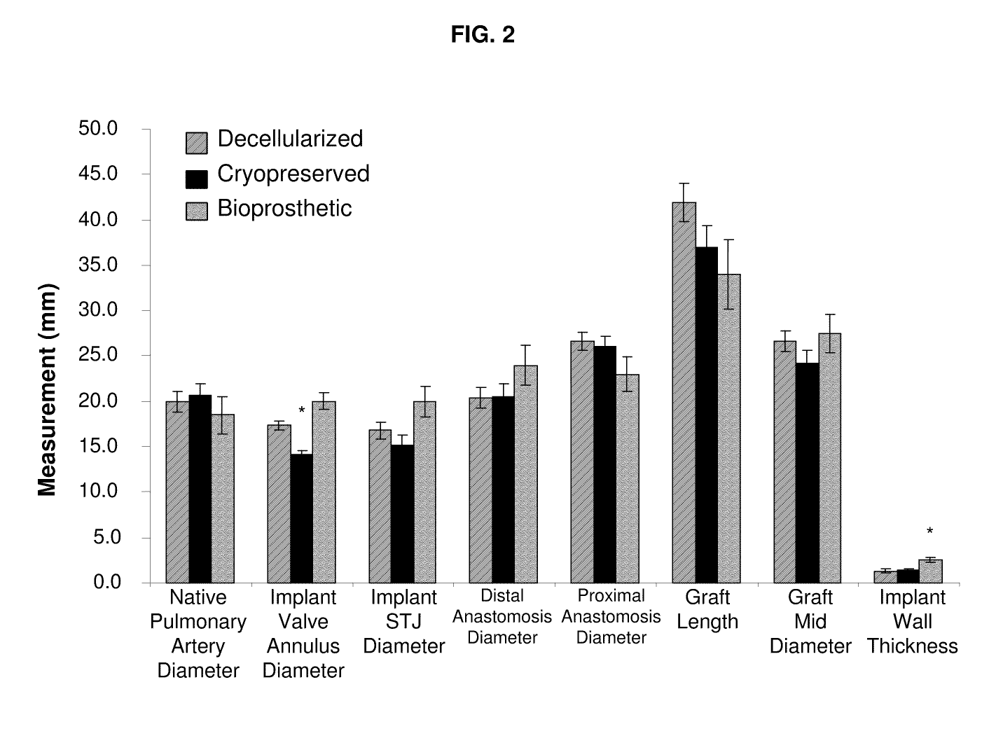

Physical characteristics of the implanted valves are presented in FIG. 2. Total implanted graft length and proximal and distal anastomosis diameters were not different between treatment groups (P=0.16; P=0.28; P=0.37). The bioprosthetic valve had a larger annulus diameter (P<0.05) and wall thickness (P=0.009) than did the cryopreserved and decellularized valves, although the size of the recipient pulmonary artery was not different between animals receiving the different valves (P=0.68).

Transesophageal Echocardiography

Immediately post-implant, no significant differences were found for mean or peak gradients between treatment groups (P=0.13 and P=0.10, respectively). Over the course of the study, EOA, EOAI, cardiac output, peak gradient and mean gradient remained constant (P>0.05 for all variables; Table 2, 3). Regurgitation was not found to change over time and was not different between treatment groups (P=0.26 and P=0.72, respectively). The cardiac index decreased over the course of the study (P<0.05). The RVOT diameter increased over the duration of the study (P=0.007) in all treatment groups (P=0.42). The bioprosthetic valve had a significantly smaller EOA (P=0.03) and a higher peak (P=0.05) and mean (P=0.01) gradient as compared to the cryopreserved and decellularized grafts. Cuspal calcification was not observed by TEE in any valve. By 8 weeks post-implant, however, leaflet excursion was restricted in the bioprosthetic valves.

Histopathology

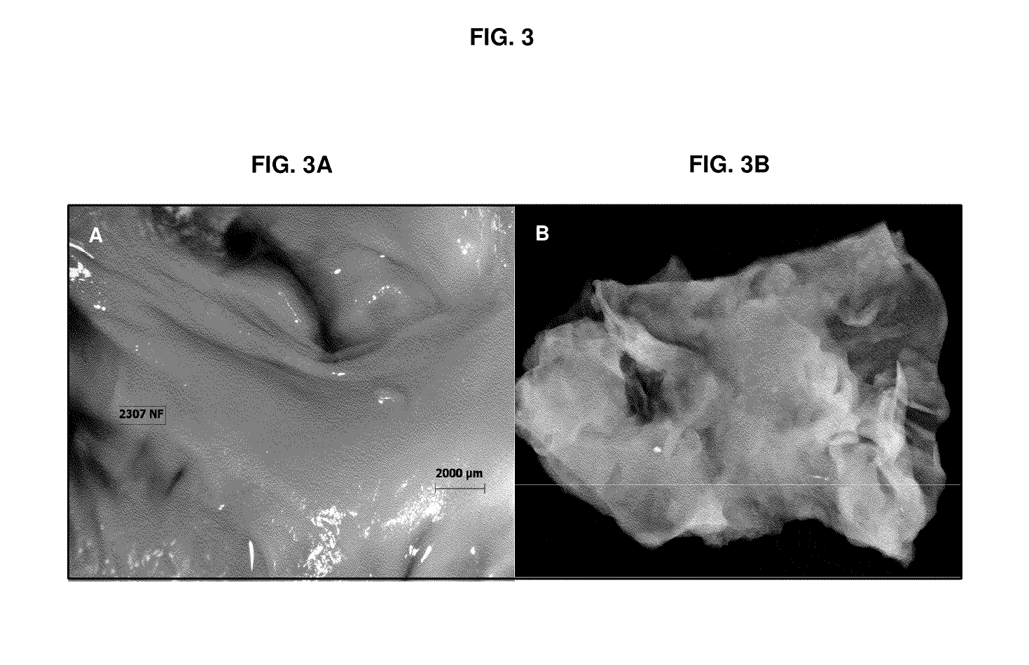

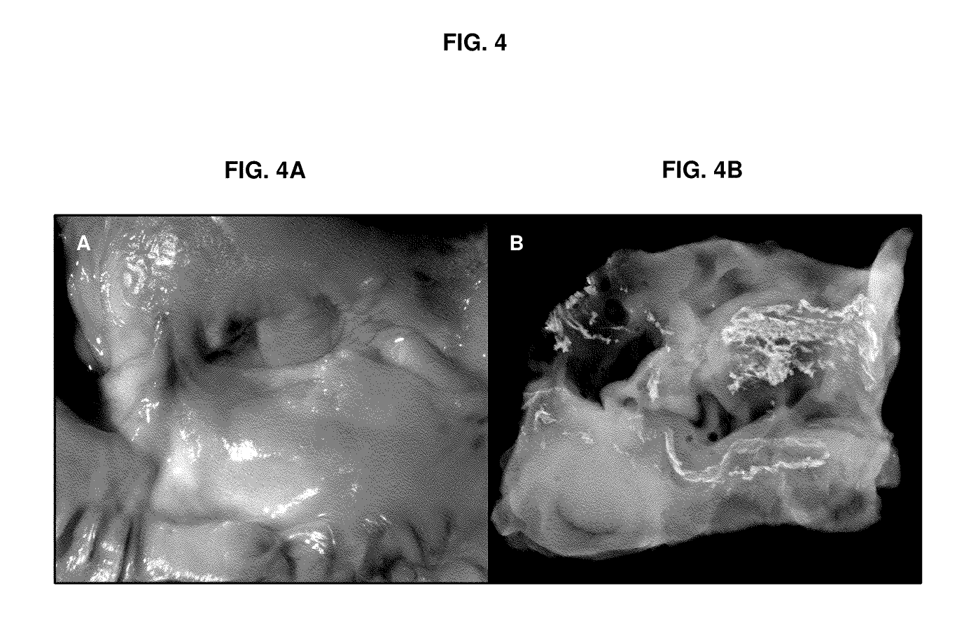

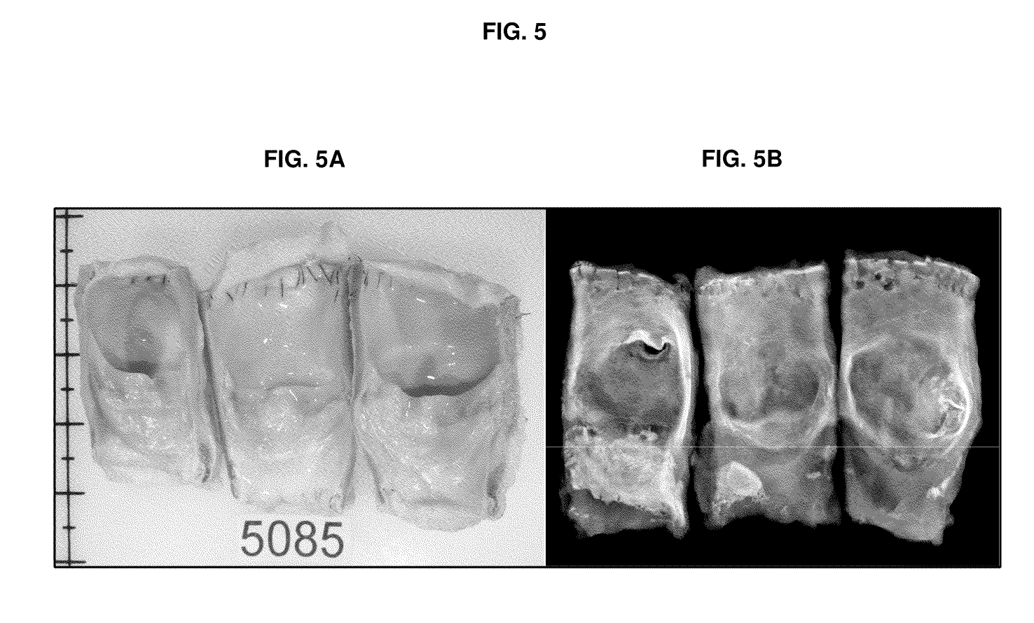

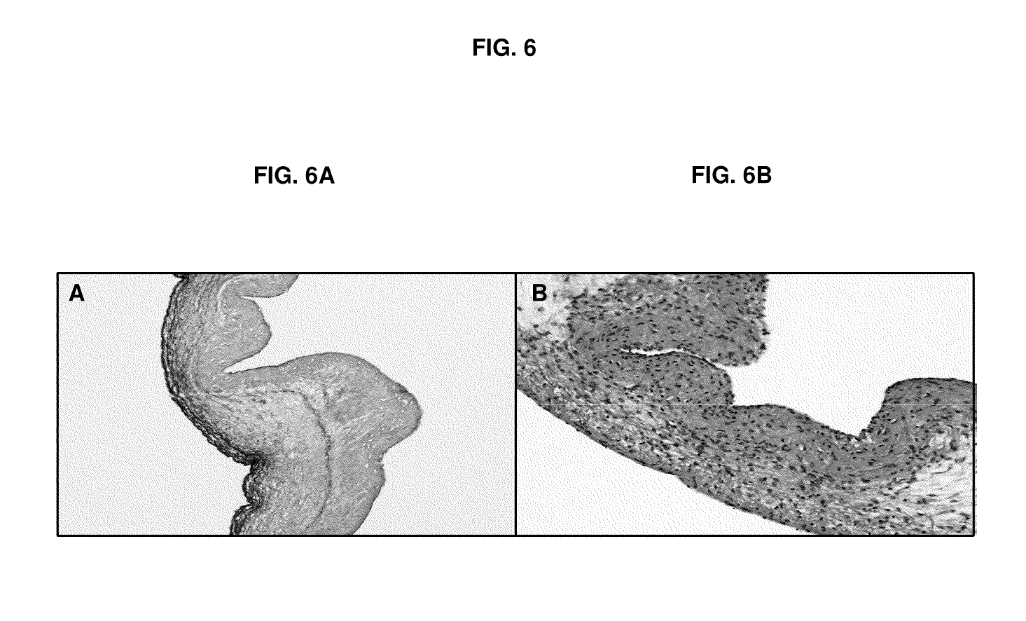

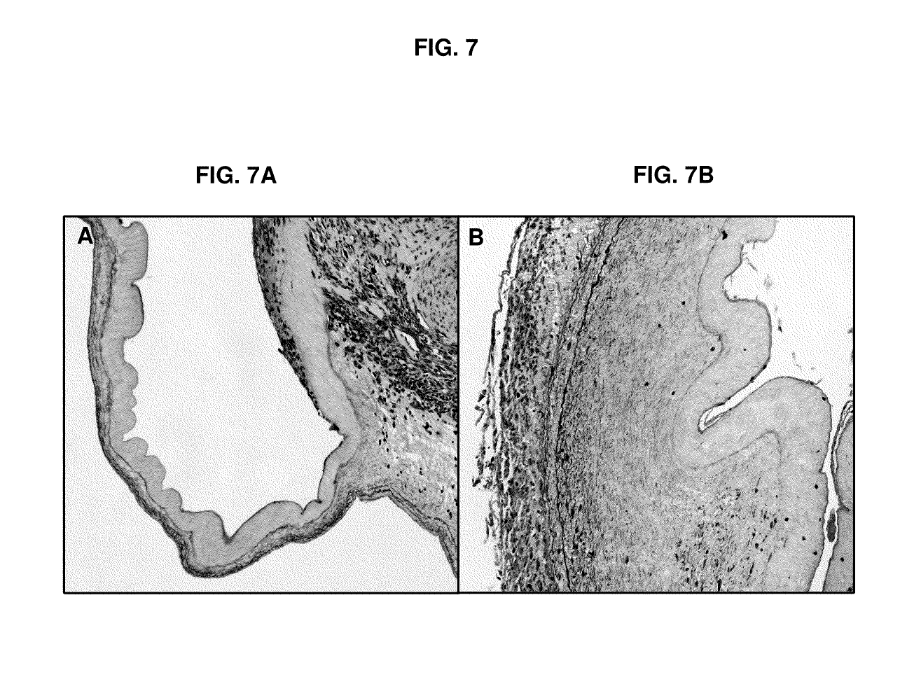

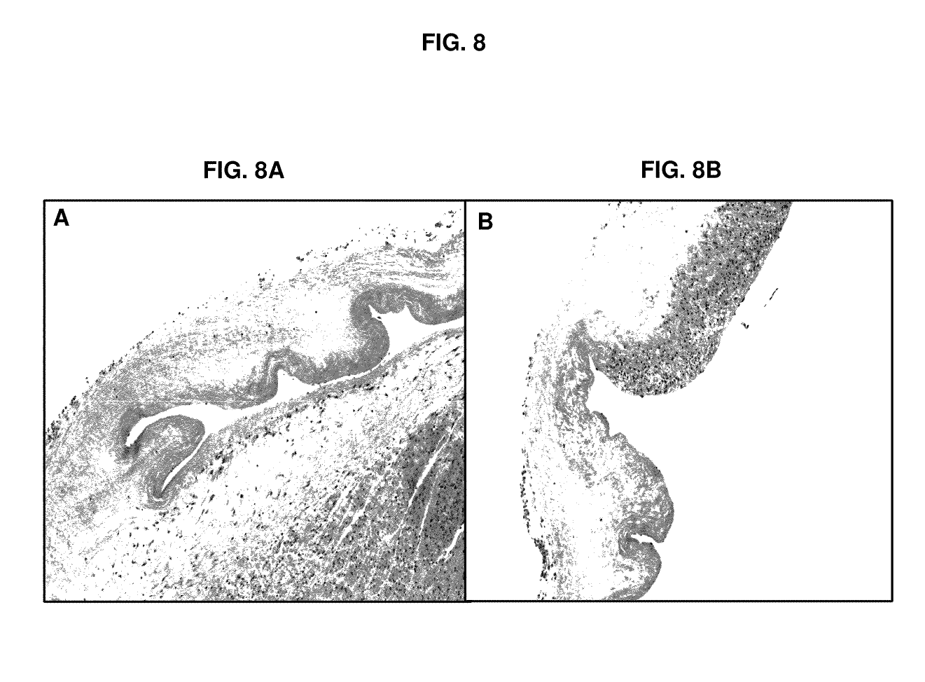

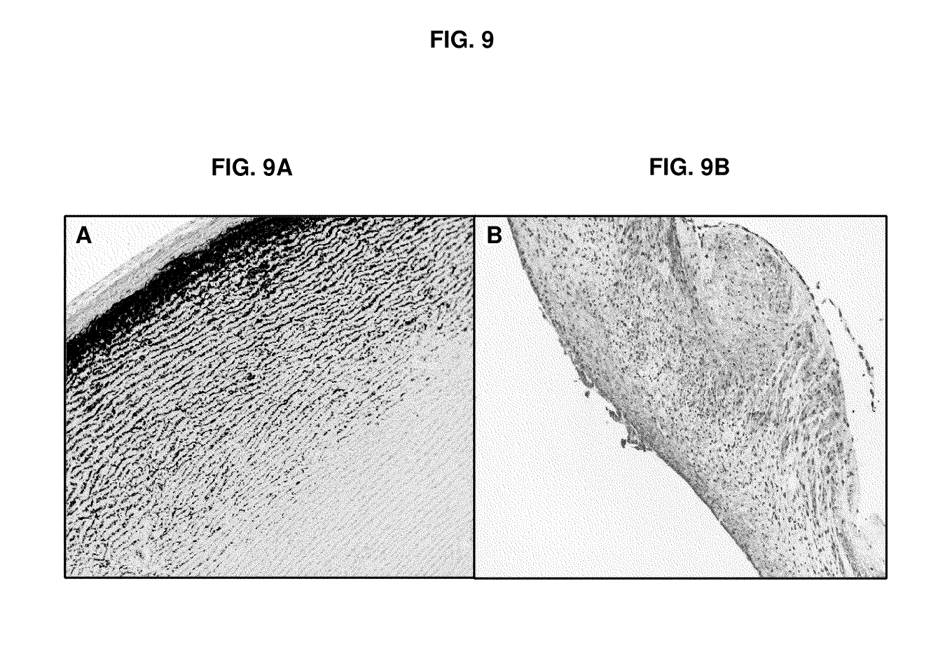

As compared to cryopreserved and bioprosthetic valves prior to implant, decellularized valves had a considerable reduction in cellularity, with no cells present in the cusp and only spotty remnants of smooth muscle cells found in the arterial wall tissue (FIGS. 3 and 4). In all three valve types, cusp morphology (ventricularis, spongiosa and fibrosa) was well preserved at 20 weeks post-implant (FIGS. 3-7). In the bioprosthetic xenograft, no autologous recellularization occurred and no inflammation was found (FIG. 9). In the cryopreserved homograft, however, cellularity was progressively lost between implant, and 20 weeks (FIG. 8). In the decellularized homograft evidence of autologous recellularization was seen in the conduit wall and extending into the base of the cusp (FIG. 7). However, no recellularization was observed in the middle and distal portions of the cusp. Re-endothelialization was variably present along the cusp of the decellularized valves. Calcification was found at the suture lines of all explanted valves and within the conduit wall of the bioprosthetic xenograft (FIGS. 3-5, 9). Calcification was not observed in the leaflets or conduit wall of either the cryopreserved or decellularized valves at explant.

Immunohistochemistry

Positive staining for desmin, vimentin and α-SMA were found in explanted decellularized valves. Additionally, positive staining for factor VIII was observed in the endothelial lining of the valve wall and cusps.

### Discussion

Decellularized pulmonary valves implanted in the RVOT of juvenile sheep were found to perform hemodynamically equally as well as cryopreserved valves and better than glutaraldehyde-fixed xenografts, and showed evidence of recellularization, which was not observed in the cryopreserved or bioprosthetic valves. These promising results favor the pursuit of the decellularized scaffold as a replacement heart valve for children and young adults. While cryopreserved homografts have traditionally been favored among bioprosthetic valve options, a growing body of research has shown that such valves, when implanted into infants and young children, may be increasingly likely to fail due to immune-related valve degeneration (Rajani et al., 1998; Mitchell et al., 1998; Vogt et al., 1999) or early homograft calcification, especially in children not matched for blood group compatibility (Christensen et al., 2004). Even in those valves that remain functional, positive panel reactive antibodies and increased HLA antibody responses are found to be associated with cryopreserved allografts (Hoekstra et al., 1997; Shaddy et al., 1996), which could have a negative impact on future valve performance or longevity (Pompilio et al., 2004). However, once the tissue is processed to remove all but trace amounts of cells or cell remnants, the antibody response is significantly reduced (Hawkins et al., 2003; Meyer et al., 2005; da Costa et al., 2005).

Decellularized valves have been found to perform equivalently to cryopreserved valves in clinical studies (Bechtel et al., 2003, 2005; Tavakkol et al., 2005; Sievers et al., 2003; Zehr et al., 2005; Costa et al., 2007), although questions exist as to whether this equivalency can be maintained over the long term (Bechtel et al., 2008). In the current study, decellularized valves showed excellent hemodynamic function, surgical handling characteristics and overall performance similar to the cryopreserved valves and were superior to the bioprosthetic valves. At implant, the cusps of decellularized valves were completely devoid of cells and no cells remained in the conduit wall, aside from small focal areas of cardiac myocytes in the sub-valvular region. The microstructure of the valve, including collagen bundles and the trilaminar architecture of the cusp remained intact.

Various methods of decellularization have been described in the literature, with the most successful being variations of detergent and enzyme extractions for both allograft (Hilbert et al., 2004) and xenograft tissues (Yang et al., 2009). Investigations into the impact of decellularization methods on valve tissue strength and structure have found that while most decellularization strategies are successful in rendering the tissue free from cells and cell debris, not all are able to balance successful cell removal with the retention of tissue integrity (Hilbert et al., 2004). Indeed, while increasing the time in which valves were exposed to decellularization agents was necessary to increase cell removal to an acceptable threshold, it was also associated with a loss of mechanical stability as measured by tensile testing (Schenke-Leyland et al., 2003). Additionally, valves decellularized with trypsin/EDTA were found to have a degraded basement membrane, disorganized collagen fibrils and weaker strength as compared to those valves treated with SD or SDS detergents (Tudorache et al., 2007) and decellularization of aortic valve leaflets with SDS, Triton-X® or trypsin was associated with the disruption of collagen crimp, increased leaflet extensibility and decreased flexural rigidity (Liao et al., 2008).

Conversely, studies have also shown no alterations to the biomechanical properties of acellular valves following decellularization with non-enzymatic digestion methods (Elkins et al., 2001; Iwai et al., 2007; Seebacher et al., 2008). Additionally, gentler decellularization methods (e.g. SDS) did not have a great impact on leaflet morphology as compared to the harsher methods (Triton-X®, trypsin; Liao et al., 2008), but it is hypothesized that some softening or loosening of the tissue structure may actually be necessary to encourage cell migration into the tissue and enhance recellularization (Liao et al., 2008). Previous research using decellularized pulmonary (Hopkins et al., 2009) or aortic (Baraki et al., 2009) valves implanted into the RVOT or left VOT of juvenile sheep indicates that proper decellularization can result in excellent hemodynamic performance and initial recellularization of the graft.

In the current study, recellularization was apparent in the explanted decellularized valves, extending from the base of the cusp through the proximal third of the leaflet. A mixture of fibroblast-like and inflammatory cells was limited to the loose spongiosa at the base of the cusp and did not extend further due to the compaction of the extracellular matrix extending the remaining two-thirds of the leaflet. Positive staining for desmin, vimentin and α-SMA is indicative of a myofibroblast-like valve interstitial cell population. The typical trilaminar leaflet structure is lost in the distal portion of the cusp in decellularized valves following implantation, with only the collagen-dense ventricularis and fibrosa found (Hilbert et al., 2004; Elkins et al., 2001). It is hypothesized that the compression of the leaflet fibers may serve as an anatomic barrier to infiltrating host cells, preventing total recellularization of the cusp (Hilbert et al., 2004). Recellularization in the devitalized valve cusp begins at the proximal anastomosis, and is a mixture of inflammatory cells and fibroblasts that migrate in waves towards the free edge of the leaflet (Hilbert et al., 2004; Elkins et al., 2001). Repopulation of the conduit is also seen in previously-decellularized tissues, consisting primarily of fibroblasts migrating from the adventitial side of the wall (Hilbert et al., 2004; Elkins et al., 2001).

The presence of an endothelial layer at explant in decellularized valves in the current study is consistent with previous reports (Erdbrugger et al., 2006). Spotty (incomplete) re-endothelialization was also seen in decellularized ovine aortic valves implanted orthotopically and explanted at 3 or 9 months (Baraki et al., 2009).

Conversely, cryopreserved valves explanted in the current study showed a marked loss of cellularity, with neither donor nor recipient cells visible in the conduit wall or cusp. Loss of cellularity in cryopreserved homografts is a common finding in explanted valves (Koolbergen et al., 2002; Vogt et al., 1999; Mitchell et al., 1995). Explanted fresh and cryopreserved allograft valves show a progressive loss of cellularity coupled with nuclear condensation, pyknosis and fragmentation, consistent with apoptosis (Hilbert et al., 1998).

In pediatric patients, cryopreserved valves explanted due to failure show acellular, calcified conduit walls containing focal regions of inflammation, primarily t-lymphocytes, indicative of an ongoing rejection response (Vogt et al., 1999). In adults, however, less inflammation is apparent, indicating that an immune-mediated rejection did not occur or occurred soon after implantation and faded away prior to explant (Vogt et al., 1999). Decellularized grafts show a reduction in inflammatory cell infiltration as compared to cryopreserved grafts (Numata et al., 2004). The presence of calcium in the conduit wall of the bioprosthetic valves is consistent with previously published reports (Herijigers et al., 1999). Calcification has also been found to occur in cryopreserved grafts implanted in the RVOT of juvenile sheep, affecting both the leaflets and conduit wall (Hopkins et al., 2009). The lack of calcification in the cryopreserved valves in the current study was unexpected but not surprising, as cryopreserved valves are used clinically with excellent results (Elkins et al., 2008; Takkenberg et al., 2009). Although the juvenile sheep is considered an exquisitely sensitive model for calcification, the extremely short warm and cold ischemic times and clinically analogous cryopreservation protocol may have provided ideal circumstances for functional, healthy cryopreserved pulmonary valves. An additional limit to the ovine model of valve replacement is the observed endocarditis that occurred across all treatment groups. Even under optimal animal husbandry conditions, a certain level of environmental pathogenic contamination of the wound may be expected. Damaged or diseased heart valves are extremely sensitive to bacterial or fungal infection and thus extra caution is required during pre-, peri- and post-operative care of the animal.

The primary goal of any decellularization strategy is to produce a valve that is 99% devoid of intact cells and cell remnants, but that retains its hemodynamic performance and is able to function similarly to the native valve. Numerous strategies exist to this end, and the protocol described herein is no exception. The results of the current study indicate that decellularized valves implanted in the pulmonary position of juvenile sheep perform equally as well as cryopreserved valves and are superior to xenograft valves and are thus suitable to serve as a scaffold for the production of a tissue engineered heart valve. Further, the valves show a marked reduction in the presence of cell remnants.

TABLE 1
Table 1. Surgical characteristics^{a} of valves implanted in the right ventricular outflow tract of juvenile sheep.

| | Treatment | | | | | | | | |
|---|---|---|---|---|---|---|---|---|---|
| | Decellularized | | Cryopreserved | | Bioprosthetic | | | | |
| Variable | median | range | median | range | median | range | X² | P > X² | Inference |
| Surgical Handling | 5 | 0 | 5 | 1 | 3.5 | 1 | 10.88 | 0.028 | Cryopreserved and decellularized |
| | | | | | | | | | valves performed better than |
| | | | | | | | | | bioprosthetic valves. |
| Ease of Implantation | 5 | 0 | 5 | 1 | 3.5 | 1 | 10.88 | 0.028 | Cryopreserved and decellularized |
| | | | | | | | | | valves performed better than |
| | | | | | | | | | bioprosthetic valves. |
| Suturability | 5 | 0 | 5 | 1 | 4 | 0 | 8.12 | 0.017 | Cryopreserved and decellularized |
| | | | | | | | | | valves performed better than |
| | | | | | | | | | bioprosthetic valves. |
| Tissue Compliance | 5 | 0 | 5 | 1 | 3 | 2 | 10.88 | 0.028 | Cryopreserved and decellularized |
| | | | | | | | | | valves performed better than |
| | | | | | | | | | bioprosthetic valves. |
| Tailorability^{b} | 5 | 0 | 5 | 0 | 4.5 | 1 | 6.46 | 0.04 | Cryopreserved and decellularized |
| | | | | | | | | | valves performed better than |
| | | | | | | | | | bioprosthetic valves. |
| Visualization^{c} | 5 | 0 | 5 | 0 | 5 | 0 | * | * | All valves performed equally well. |
| Bleeding^{d} | 5 | 0 | 5 | 0 | 5 | 0 | * | * | All valves performed equally well. |
| Kink Resistance | 5 | 0 | 5 | 0 | 5 | 0 | * | * | All valves performed equally well. |

^{a}Surgical characteristics were scored on a scale of 1 (poor) to 5 (excellent) by the surgeon immediately following completion of surgery.
^{b}Ease of tailoring to suit patient anatomy.
^{c}Ease of visualization of surgical field.
^{d}Ease of control of suture line bleeding.
* Chi square and associated p-values could not be calculated as responses were identical for all experimental units in each treatment group.

TABLE 2
Table 2. Transesophageal echocardiography measurements at 0, 8, 20, 32 and 52 weeks post-implant for juvenile
sheep receiving either a decellularized, cryopreserved or bioprosthetic replacement pulmonary valve.

| | | | EOA | | EOAI | | Mean Gradient | | Peak Gradient | | CO | | CI^{e} | |
|---|---|---|---|---|---|---|---|---|---|---|---|---|---|---|
| | | | (cm²) | | (cm²/m²) | | (mm Hg) | | (mm Hg) | | (L/min) | | (L/min/m²) | |
| Time | n = | Implant Type | Mean | SEM | Mean | SEM | Mean | SEM | Mean | SEM | Mean | SEM | Mean | SEM |
| 0 wk | 5 | cryopreserved | 1.76 | 0.31 | 1.88 | 0.36 | 5.25 | 2.26 | 8.82 | 4.1 | 5488.3 | 1123.8 | 5.92 | 0.49 |
| | 2 | bioprosthetic | 1.51 | 0.43 | 1.78 | 0.46 | 8.5 | 2.62 | 13.03 | 4.77 | 6.42 | 1.44 | 7.40 | 0.67 |
| | 7 | decellularized | 2.39 | 0.29 | 2.47 | 0.34 | 3.06^{a} | 2.18 | 3.99^{a} | 3.98 | 4.63 | 1.06 | 5.61 | 0.49 |
| 8 wk | 5 | cryopreserved | 2.11^{b,c} | 0.27 | 2.16 | 0.33 | 3.17 | 2.25 | 5.55 | 4.09 | 4.21 | 0.92 | 4.24 | 0.45 |
| | 2 | bioprosthetic | 1.31^{a,c} | 0.34 | 1.44 | 0.38 | 5.82 | 2.5 | 8.55 | 4.56 | 3.87 | 1.04 | 4.03 | 0.61 |
| | 7 | decellularized | 2.65^{b} | 0.25 | 2.53 | 0.31 | 2.6 | 2.9 | 4.87 | 3.98 | 5.11 | 0.90 | 4.81 | 0.43 |
| 20 wk | 5 | cryopreserved | 2.41 | 0.41 | 2.06 | 0.38 | 3.48 | 2.24 | 6.56 | 4.06 | 477 | 0.98 | 4.06 | 0.48 |
| | 2 | bioprosthetic | 2.52 | 0.59 | 1.95 | 0.49 | 9.23^{a} | 2.44 | 13.06^{a} | 4.43 | 5.60 | 1.18 | 4.33 | 0.65 |
| | 7 | decellularized | 2.61 | 0.36 | 2.2 | 0.36 | 2.37 | 2.18 | 4.31 | 3.97 | 5.13 | 0.95 | 4.30 | 0.46 |
| 32 wk^{d} | 3 | cryopreserved | 2.64 | 0.24 | 2.09 | 0.35 | 2.9 | 2.27 | 5.61 | 4.11 | 5.28 | 0.94 | 4.03 | 0.44 |
| | 3 | decellularized | 2.81 | 0.23 | 2.18 | 0.34 | 1.27 | 2.21 | 3.05 | 4.02 | 4.57 | 0.94 | 3.55 | 0.47 |
| 52 wk | 3 | cryopreserved | 2.92 | 0.33 | 1.85 | 0.38 | 2.28 | 2.26 | 4.13 | 4.09 | 4.48 | 1.01 | 2.73 | 0.48 |
| | 3 | decellularized | 2.87 | 0.32 | 1.74 | 0.37 | 1.94 | 2.21 | 4.04 | 4.01 | 5.21 | 1.02 | 3.22 | 0.51 |

^{a,b,c}Data within columns with different superscripts are significantly different (P < 0.05) within each time period. EOA, effictive orifice area; EOAI, EOA normalized to body surface area; CO, cardiac output; CI, cardiac index
^{d}Four decellularized, two cryopreserved and two bioprosthetic valves were explanted at 20 wk
^{e}Significant effect of time, P < 0.05. Treatment by time, N.S.

TABLE 3
Table 3. Left-sided cardiac output (CO) and cardiac index (CI) values
measured by transesophageal echocardiography at 0, 8 20, 32 and 52
weeks post-implant for juvenile sheep receiving either a decellularized,
cryopreserved or bioprosthetic replacement pulmonary valve

| | | | Left-sided | | Left-sided CI^{a} | |
|---|---|---|---|---|---|---|
| | | Implant | CO (L/min) | | (L/min/m²) | |
| Time | n = | Type | Mean | SEM | Mean | SEM |
| 0 wk | 5 | cryopreserved | 3421 | 651.6 | 2859.4 | 546.3 |
| | 2 | bioprosthetic | 4269.2 | 950 | 3947.9 | 808 |
| | 7 | decellularized | 4420.3 | 574.2 | 3655.9 | 477.5 |
| 8 wk | 5 | cryopreserved | 3338.1 | 473.4 | 2848.6 | 354.4 |
| | 2 | bioprosthetic | 3435.5 | 587.7 | 3248.5 | 439.3 |
| | 7 | decellularized | 3535 | 403.1 | 2836.5 | 303.2 |
| 20 wk | 5 | cryopreserved | 4533.7 | 659.3 | 3235.4 | 493.8 |
| | 2 | bioprosthetic | 5697.2 | 959.1 | 4224 | 717.4 |
| | 7 | decellularized | 4261.1 | 613.7 | 3026.1 | 459.4 |
| 32 wk^{b} | 3 | cryopreserved | 3901 | 552.1 | 2601.4 | 339.1 |
| | 3 | decellularized | 3811 | 554.5 | 2456.4 | 341.3 |
| 52 wk | 3 | cryopreserved | 3945.4 | 402.3 | 2521 | 333.7 |
| | 3 | decellularized | 5167.3 | 405.4 | 3075.4 | 335.9 |

^{a}CI, cardiac index (CO normalized to body surface area)
^{b}Four decellularized, two cryopreserved and two bioprosthetic valves were explanted at 20 wk

### Example 2

This examples illustrates one embodiment of the decell process of the present invention.

### Materials and Methods

### Solutions Used:

a. Triton X®-100 (Triton): 0.05% Triton X®-100 solution a 1:2000 dilution derived from 100% Triton X®-100 detergent (Sigma T8787) in ddH₂O. Each valve will need 200 mL of this solution, which can be made ahead of time.
   For 2 L use 1 mL 100% Triton-X®, 1999 mL ddH₂O.
b. N-lauroylsarcosine Sodium Salt Solution (NLS): 1% NLS Solution a 1:20 dilution derived from 20% Sodium Laureth Sulfate (Sigma—L7414) in ddH₂O. Each valve will need 200 mL of this solution, which can be made ahead of time.
   For 2 L use 100 mL 20% NLS, 1900 mL ddH₂O
c. Hypertonic Salt Solution (HSS): 1% NaCl (Fisher—BP358-1), 12.5% D-Mannitol (Sigma—M9647), 5 mM MgCl₂ (Sigma—M2643), 500 mM KCl (Sigma P4504) in NS (Normal Saline). Each valve will need 200 mL of this solution which can be made ahead of time.
   For 2 L use 2 L NS, 18 gm NaCl, 2.03 gm MgCl₂, 74.3 gm KCl, 250 gm Mannitol.
d. 2×Saline Mannitol Solution (SMS): 1% NaCl (Fisher—BP358-1), 12.5% D-Mannitol (Sigma—M9647). Each valve will need 200 mL of this solution which can be made ahead of time.
   For 2 L use 2 L NS, 18 gm NaCl, 250 gm Mannitol.
e. RNA-DNA Enzyme Extraction Buffer (BENZ): 12.5 KU of Benzonase® (Sigma—E1014) per 200 mL ddH₂O, 8 mM MgCl₂ (Sigma—M2643), pH to 8.0 using diluted NH₄OH (˜100 μL needed of 1M solution). Each valve will need 400 mL of this solution which should be made on the day of use.
   For 400 mL use 400 mL ddH₂O, 1 vial Benzonase® (25 KU), 650 mg MgCl2 (Sigma—M2643)
f. Organic Solvent Extraction Buffer (EtOH): 2:5 dilution of ethyl alcohol 200 proof (Sigma—459836) in ddH₂O—40% v/v solution. Each valve will need 200 mL of this solution, which can be made ahead of time. For 2 L use 800 mL ethanol, 1200 mL ddH₂O

Valves were dissected in a laminar flow safety cabinet using sterile technique and stored individually, in 200 mL of preprocessing storage solution in sterile 250 mL jars for 72 hours at 4° C.

On Day One of processing the detergent and osmotic shock sequences were performed. The 250 mL flasks containing the valve tissue were each filled with 200 mL HSS with one heart valve in each jar. Flasks were then placed on a rocker plate for 2 hours at 220 RPM at room temperature. The valves were then washed for 3 hours in Triton® at 220 RPM at room temperature at a temperature of 21° C. Each wash or rinse was conducted in a new sterile 250 mL flask and transfer was completed under a sterile laminar flow hood. A rinse was then performed on the valves one time for 10 minutes in ddH₂O at 220 RPM at room temperature. The valves were then washed for 2 hours in HSS at 220 RPM at room temperature. Another rinse was performed for 1 hour in ddH₂O at 220 RPM at room temperature. The valves were then washed for 3 hours in Triton® at 220 RPM at room temperature. Next, a RNA-DNA enzyme extraction was performed. A flask containing sterilized BENZ at a pH of 8.0 was used for the extraction and the valves were transferred into the BENZ solution to shake on a rocker plate at 220 RPM at 37° C. overnight.

On Day Two of Processing, the valves were rinsed for 1 hour in ddH₂O at 220 RPM at room temperature, washed, and then placed in NLS solution on a rocker plate O/N at 220 RPM at room temperature.

On Day Three of Processing, an organic extraction was performed. Valves were rinsed once for 4 hours in ddH₂O at 50 RPM at room temperature. Next, an extraction was completed using ethyl alcohol. For the extraction, the valves were rinsed for 30 minutes with 40% EtOH at 50 RPM at room temperature. After the extraction, an ion exchange detergent residual extraction for dual chamber was set up. FIG. 1 illustrates how the exchange chamber was assembled. 50 gm of each type of bead were used. The beads were soaked in EtOH for 5 minutes and then quickly rinsed in ddH₂O. The beads were then aseptically added to and 8 L spinner flask. The valves were then aseptically added to the 10 L bioreactor flask. Throughout this process, all connections were sprayed down with 70% EtOH as needed. The spinner flasks were then filled with 7 L ddH₂O by connection ports to 10 L reservoir via peristaltic pump and silicone tubing. Both stir plates were spun at 60 RPM and the peristaltic pump was set to 48 RPM (150 mL/min, max. setting).

On Day Four of Processing, a Mannitol wash or soak was performed. The wash or soak was carried out for those valves which were not immediately being placed into the post-decellularization storage solution for immediate use. For those valves placed in the wash or soak, they were washed or soaked for 2 hours in 200 mL SMS on a rocker plate at 50 at room temperature. A new sterile 250 mL flask with 200 mL post-decellularization storage solution was used to place each valve in for storage purposes.

### Results and Conclusions

The decellularization process produced a heart valve with better biomechanical properties, less calcification, little to no dsDNA left in the valve tissue, and better elasticity properties than heart valves that were cryopreserved or decellularized using a different method other than that of the present invention.

### Example 3

This example illustrates the multi-anionic detergent/enzyme pH controlled reciprocating osmolality mammalian heart valve decellularization method for the creation of ECM Scaffold to be used for heart valve tissue engineering.

### Materials and Methods

Hearts were aseptically harvested during multi organ donor harvest. The transport solution were sterile lactated ringers or PBS solution with 4× amphotericin B*=4 ug/ml and 4× penicillin/streptomycin*=400 IU/mL) were provided in advance. *standard tissue culture medium concentrations were Ampho (250 ug/mL) at 8 ug/mL=2 mL/500 mL and Pen/Strep (10 K IU/mL) at 100 IU/mL=5 mL/500 mL.

The valves were dissected in a laminar flow safety cabinet using sterile technique and stored, individually, in sterile 250 mL bottles with fresh transport solution at 4° C. for a maximum of 72 hours. All solutions and solvents used were sterile. Next a muscle shelf debridement protocol was performed.

On the first processing day, the valves were placed in 1% of HSS (v/v) solution for 3 hours on a rocker plate at 25 RPM at a temperature of 37° C. with dilute 20% INLS to 1%+200 mL ddH₂O (double deionized water) in a 250 mL flask (wide mouth sterile cap). Next, the valves were rinsed in ddH₂O three times on a rocker plate at 40 RPM for approximately 1 minute each to rinse at a temperature of 21° C. The valves were then placed in a hypertonic salt solution for 2 hours on a rocker plate at 25 RPM. The hypertonic salt solution contained 2.00 gm of NaCl in 200 mL 0.9% physiologic saline solution with approximately 2.00 mg MgCl₂ (5 mM) (MW=203.03), 9.00 gm KCl (500 mM) (MW=74.56), and 12.5 gm Mannitol (25% solution). The valves were then washed again three times on a rocker plate at 25 RPM for 1 hour each wash at a temperature of 21° C. The valves were then placed in an overnight solution of 10K U Benzonase® in 200 mL ddH₂O with approximately 400 mg MgCl₂ (10 mM) and 20 uL NH₄OH at a pH of 9.0.

On the second processing day, a detergent extraction was performed. The valves were washed three times in ddH₂O on a rocker plate at 25 RPM for 1 hour each wash at 21° C. During the second wash, alpha galactosidase 1 u/200 cc ddH₂O (recombinant and the pH was adjusted to a level between 6.5-7.4 at a temperature of 30° C. A 1% NLS solution comprising 2.0 gm NLS in 200 mL ddH₂O at 21° C., was used as an overnight storage solution for the valves.

On the third processing day, an organic solvent extraction was performed. The valves were once again washed twice in ddH₂O on a rocker plate at 25 RPM for 1 hour each wash at 21° C. The valves were then transferred into 4 L beakers with a cover to be decanted and react in new 250 mL sterile individual bottles. Covering each valve was V/V 25% ethyl alcohol solution for 4 hours on a rocker plate at 25 RPM at 21° C. The ethyl alcohol solution was comprised of 100% ethyl alcohol (200 proof) in 2 L ddH₂O, Next an ion exchange detergent residual extraction was performed. Two columns were set up with a new reservoir (4 L flask). Beads from Sigma-Aldrich were used. 4 L ddH₂O+amberlite in a sterile flask reservoir of the decell bioreactor system. Two other in-line columns packed with beads were used for the two other extractors. In the columns were IWT® TMD-8 hydrogen and hydroxide form #378593-500G, Sigma-Aldrich, XAD 15 nonionic hydrophobic, and Dowex monosphere 550 A (UPW ammonic and cationic). Additionally 10 gm in 2 L ddH₂O IWT (100 gm/10 L), 10 gm XAD-16 Amberlite (100 gm/10 L), and 10 gm Bio-beads Dowex (100 gm/10 L) were added. The valves were then placed into a 10 L bioreactor. After valves were transferred into the decell chambers, the reservoir was attached for continuous exchange overnight on a magnetic stirrer plate at 15 RPM. The decell chambers were loaded with sterile ddH₂O. The extraction was performed at a temperature of 21° C. for all three exchanges. The flow rate was 30 cc/minute with a max of 45 cc/minute.

On day 4 of processing, the reservoir flasks were changed out and a u-tube trap was placed on the decell chamber/bioreactors. A new sterile flask, trap, and tubing were used for each. The ddH₂O was removed to minimize bead loss or new beads were placed in the reservoir flask. The reservoir bottles were changed to fresh sterile ddH₂O to the reservoir decell bioreactor at 25 RPM stirring at 15 RPM rotor settings in the bioreactor for 4 hours. The valves were then washed with ddH₂O. A sterile flask was used as a container into which the valves were transferred. The sterile flask was covered and contained a soak or rinse of ddH₂O. The valves were decanted in the ddH₂O for 15 minutes, then decanted in PBS for 15 minutes and then decanted for an hour in ddH₂O at 21° C. Finally, the valves were transferred from sterile decell valves to new, sterile individual bottles to store at a temperature of 4° C. (wet ice) for storage refrigeration for about or less than 3 weeks. The valves were stored in post-decellularization storage solution containing 250 mL sheep (or species specific papio, human) serum or plasma, 4 X Amphotericin B (250 ug/mL) 4 mL/250 mL, 2× Pen/Strep (10 KIU/mL) 5 mL/250 mL, Levaquin (25 mg/mL) 0.5 mL/250 mL, and Vancomycin (2.5 ug/mL=0.5 mL/250 mL).

### Results and Conclusions

The decellularization process removes cells and cell debris (cell remnants) from the tissue and does so without a harmful effect on the heart valve. The tissue preparation allows for donor cells to be easily transferred and grown in the decellularized tissue. The decellularization process also prevents the calcification of the heart valve, leading to additional problems.

### Example 4

This example illustrates the tests for toxicity on the decellularized valves.

### Materials and Methods

Ovine valvular tissues were used. The extraction vehicle was single strength Minimum Essential Medium (MEM) supplemented with 5% serum and 2% antibiotics (1×MEM). To prepare the tissue, a 9.9 g portion of the test article was covered with 50 mL of 1×MEM. A single preparation was extracted with agitation at 37° C. for 24 hours. High density polyethylene was used as a negative control. For the reagent control preparation, a single aliquot of 1×MEM was used. For the positive control preparation, tin stabilized polyvinylcholoride was used. The test system used was mouse fibroblast cells which were propagated and maintained in open wells containing single strength Minimum Essential Medium supplemented with 5% serum and 2% antibiotics (1×MEM) in a gaseous environment of 5% carbon dioxide. For this study, 10 cm² wells were seeded, labeled with passage number and date, and incubated at 37° C. in 5% CO₂ to obtain sub-confluent monolayers of cells prior to use.

Triplicate culture wells were selected which contained a sub-confluent cell monolayer. The growth medium contained in the triplicate cultures was replaced with 2 mL of the test extract. Similarly, triplicate cultures were replaced with 2 mL of the reagent control, negative control, and positive control. The wells were incubated at 37° C. in 5% CO₂ for 48 hours. Following incubation, the cultures were examined microscopically (100×) to evaluate cellular characteristics and percent lysis. The color of the test medium was observed. A color shift towards yellow was associated with an acidic pH range and a color shift towards magenta to purple was associated with an alkaline pH range. Each culture well was evaluated for percent lysis and cellular characteristics according to the following table.

TABLE 4

| | Reac- | |
|---|---|---|
| Grade | tivity | Conditions of All Cultures |
| 0 | None | Discrete intracytoplasmic granules; no cell lysis |
| 1 | Slight | Not more than 20% of the cells are round, loosely |
| | | attached, and without intracytoplasmic granules; no |
| | | extensive cell lysis and empty areas between cells |
| 2 | Mild | Note more than 50% of the cells are round and |
| | | devoid of intracytoplasmic granules; no extensive |
| | | cell lysis and empty areas between cells |
| 3 | Moderate | Not more than 70% of the cell layers contain |
| | | rounded cells or are lysed |
| 4 | Severe | Nearly complete destruction of the cell layers |

### Results and Discussion

TABLE 5
Reactivity Grades for Elution Testing

| | | Percent Cells | | | |
|---|---|---|---|---|---|
| | Percent | Without Intracyto- | Percent | | Reac- |
| Well | Rounding | plasmic Granules | Lysis | Grade | tivity |
| Test 1(a) | 0 | 0 | 0 | 0 | None |
| Test 1(b) | 0 | 0 | 0 | 0 | None |
| Test 1(c) | 0 | 0 | 0 | 0 | None |
| Negative | 0 | 0 | 0 | 0 | None |
| Control | | | | | |
| 1(a) | | | | | |
| Negative | 0 | 0 | 0 | 0 | None |
| Control | | | | | |
| 1(b) | | | | | |
| Negative | 0 | 0 | 0 | 0 | None |
| Control | | | | | |
| 1(c) | | | | | |
| Reagent | 0 | 0 | 0 | 0 | None |
| Control | | | | | |
| 1(a) | | | | | |
| Reagent | 0 | 0 | 0 | 0 | None |
| Control | | | | | |
| 1(b) | | | | | |
| Reagent | 0 | 0 | 0 | 0 | None |
| Control | | | | | |
| 1(c) | | | | | |
| Positive | 100 | 100 | 100 | 4 | Severe |
| Control | | | | | |
| 1(a) | | | | | |
| Positive | 100 | 100 | 100 | 4 | Severe |
| Control | | | | | |
| 1(b) | | | | | |
| Positive | 100 | 100 | 100 | 4 | Severe |
| Control | | | | | |
| 1(c) | | | | | |

Under the conditions of this study, the 1×MEM text extract showed no evidence of causing cell lysis or toxicity. The 1×MEM test extract met the requirement of the test since the grade was less than a grade 2 (mild toxicity). In conclusion, there was no cytotoxicity indicated for the 1×MEM.

### Example 5

This example illustrates another embodiment of the decellularization method of the present invention.

### Materials and Methods

Valves were dissected in a laminar flow safety cabinet using sterile technique and stored individually, in 200 mL of preprocessing storage solution in sterile 250 mL jars for 72 hours at 4° C. On Day 1 of processing a detergent and osmotic shock sequence was performed. The 250 mL flasks were filled with 200 mL HSS and the valves inserted into individual flasks. The flasks were placed on a rocker plate for 3 hours at 220 RPM. The valves were then washed with Triton® for 3 hours at 220 RPM. Each wash or rinse was conducted in a new flask. The valves were then rinsed in ddH₂O three times for 15 minutes each time, at 220 RPM. The valves were washed next in 200 mL sterile HSS on a rocker plate for 220 RPM. A rinse of ddH₂O for 1 hour was then performed on a rocker plate at 220 RPM at room temperature. The valves were then washed again in Triton® for 3 hours on a rocker plate at 220 RPM. The valves were then transferred to flasks containing sterilized BENZ. The flasks were then incubated overnight on a rocker plate at 220 RPM at 37° C.

On Day 2 of processing an enzyme treatment and second detergent wash were performed. The valves were rinsed for 1 hour in ddH₂O on a rocker plate at 220 RPM at room temperature. The next wash was with NLS solution on a rocker plate overnight with the addition of BENZ to each flask incubated on a rocker plate at 220 RPM at room temperature.

On Day 3 of processing, the valves were rinsed three times for 2 hours at a time in ddH₂O on a rocker plate at room temperature. The next rinse was in ddH₂O overnight at 220 RPM at room temperature.

On Day 4 of processing, an organic solvent extraction was performed. First, an ethyl alcohol extraction was performed, where the valves were rinsed with 200 mL of 40% EtOH solution on a rocker plate at 220 RPM at room temperature. Next, an ion exchange detergent residual extraction was set up for each valve. Nylon mesh pouches containing 30 g of beads (Amberlite, Dowex, IWT) were sealed with a crimper. The pouches were then soaked in 100% EtOH for 3-5 minutes each and then rinsed with ddH₂O in a hood before placing the valves into a 6 L microcarrier spinner flask. Each spinner flask was then filled with 7 L ddH₂O by connecting ports to a 10 L reservoir via peristaltic pump and silicone tubing. The reservoir was then raised above the flask if gravity was required to prime pump. All connections were sprayed with 70% EtOH to disinfect. The valves were placed in individual metal cages and aseptically inserted into a spinner flask which was spun at 100 RPM for 4 hours overnight.

On Day 5, the ddH₂O was removed from the spinner flasks by reconnecting the peristaltic pump and pumping into empty reservoir. This was repeated and run for 4 hours. The valves were then soaked for 15 minutes in 200 mL ddH₂O on a rocker plate at 100 RPM at room temperature. The next rinse was in 200 mL normal saline for 15 minutes on a rocker plate at 100 RPM at room temperature. Another rinse of 200 mL ddH₂O was then performed for an hour on a rocker plate at 100 RPM at room temperature. The valves were then soaked for 3 hours in 200 mL SMS on a rocker plate at 100 RPM for 1 hour. All valves were then transferred to new sterile flasks with 200 mL post decellularization storage solution.

### Results and Discussion

The decellularization process removes cell debris from the tissue and does so without a harmful effect on the heart valve. The tissue preparation allows for donor cells to be easily transferred and grown in the decellularized tissue. The decellularization process also prevents the calcification of the heart valve, leading to additional problems.

### Example 6

This example illustrates that tissue decellularized according to the methods of the present invention has little or no dsDNA present within the scaffold.

### Materials and Methods

Test articles were dissected into leaflet, sinus and wall regions. Duplicate samples per leaflet, sinus and wall were weighed per test article. Double strand DNA was isolated using the Qiagen DNeasy Blood & Tissue Kit. Triplicate portions of the isolated dsDNA samples were prepared using the Molecular Probes Quant-iT dsDNA Assay Kit-High Sensitivity. The test control (wall) was prepared and analyzed in the same manner.

The prepared samples were analyzed using the BioTek Synergy HT Fluorometer. DNA was extracted from ˜25 mg of tissue using Qiagen DNeasy Blood & Tissue Kit. Triplicate portions of the extracted DNA were prepared for analysis using Molecular Probes Quanti-iT dsDNA Assay Kit-High Sensitivity. The prepared solutions were analyzed using a BioTek Synergy HT Fluorometer.

### Results and Conclusions

### Data Analysis

The amount of dsDNA per wet weight of tissue for control article, cryopreserved valves and omega decellularized valves were calculated. Results were reported for the average dsDNA concentration. Double stranded DNA was detected in all cryopreserved pulmonary valve portions (leaflet, sinus and wall). The results for the cryopreserved valve are consistent with the test control. Double stranded DNA was not detected in the omega decellularized leaflet and sinus regions of the valves. A very low amount of dsDNA was detected in two wall regions of the omega decellularized valves, but only in one of three determinations per test article sample.

TABLE 6
Average dsDNA Concentration

| | dsDNA/wet weight | | |
|---|---|---|---|
| Tissue Treatment | (μg/mg) | Stdev | n |
| Cryopreserved | 0.0875 | 0.0257 | 18 |
| Omega Decellularized | 0.0001 | 0.0005 | 48 |
| Porcine Control | 0.1287 | 0.0083 | 2 |

Three cryopreserved ovine pulmonary and eight omega decellularized ovine pulmonary valves were analyzed for dsDNA. Duplicate portions of native porcine pulmonary wall, stored at −80° C., and were analyzed for dsDNA as test controls. Double stranded DNA was detected in all cryopreserved pulmonary valve portions (leaflet, sinus and wall) and test controls. The results for the cryopreserved valve are consistent with the test control. The average dsDNA for cryopreserved valves and test control wall are 0.0875±0.0257 μg/mg and 0.1287±0.0083 μg/mg, respectively. Double stranded DNA was not detected in the omega decellularized leaflet and sinus regions of the valves. A very low amount of dsDNA was detected in two wall regions of the omega decellularized valves, but only in one of three determinations per test article sample. The average dsDNA for omega decellularized valves are 0.0001±0.0005 μg/mg.

### Example 7

This example illustrates a comparison of differential calorimetry scanning (DSC) analysis on decellularized and cryopreserved heart valves.

### Materials and Methods

Two decellularized and two cryopreserved valves were dissected into 3 cusps according to the valve allocation matrix. The cusp for DSC was further dissected into leaflet, sinus, and arterial wall. Eight tissue specimens from the decellularized tissue and 10 from the cryopreserved were cut out of each leaflet, sinus, and arterial wall and their mass was recorded as a wet tissue weight. The samples were approximately 5 mg samples. They were placed in aluminum sample pans weighed again and taken to the DSC. These specimens were then tested in the DSC by heating the tissue from 40° C. to 90° C. by 5° C./min to generate thermograms. From the thermograms the onset temperature, peak temperature and enthalpy were collected. The pans were punctured and placed in an oven to dry the tissue. The dry pan weight was measured and recorded so the moisture content could be calculated.

The following equipment and materials were used: Perkin-Elmer Diamond DSC, Perkin-Elmer stainless steel sample pans, Perkin-Elmer sealing press, Perkin-Elmer vacuum pen, Precision oven, Mettler-Toledo mass balance, 2 vol % Contrad 70, Ethanol, Deionized water, Dissected tissue samples, Scalpel or razor blade, Weighing boat, Forceps, Hammer, and Small punch.

### Hardware and Software Startup

The Perkin-Elmer Diamond DSC was turned on first. The Pyris software (PerkinElmer, Waltham, Mass.) was open using the Pyris Manager shortcut on the desktop. From the Window dropdown menu, the Instrument Viewer was selected. The furnace was then cleaned to improve the quality of thermograms. Next, the sample lid and cover were opened and the sample and reference pans were removed. Both the sample lid and cover were left open while performing the furnace cleaning operation. The Diamond DSC Control Panel was used to initiate the flow of nitrogen around the sample chamber. Next, the Clean Furnace button on the control pane was clicked and the cleaning operation began. Afterwards, the sample lid was covered and placed into the Intercooler II. The Cover Heater was then turned on. The DSC was allowed to stabilize for 30 min under these conditions before the calibration procedure was completed or any thermograms were collected. Calibration of the DSC was performed daily.

Next, the thermograms were collected. To prepare the samples, samples were cut from leaflet, sinus wall and vessel wall tissue using a scalpel or razor blade. Samples were sized such that they could be placed in a DSC sample pan without making contact with the side walls or top cover (10-20 mg wet weight). The sample was placed on filter paper for one minute to remove excess water, flipping the sample every 10 seconds. The empty sample pan, including top and bottom potions of the pan, was emptied. Next, the tissue sample was placed in the bottom portion of a clean sample pan. The sample was placed at the bottom portion of the pan in the sealing press pan holder. Next, the pan holder was assembled and mounted on the sealing press. The pan was sealed slowly by moving the lever arm. The sample pan was then transferred from the pan holder to the micro-balance using a vacuum pen and the weight of the assembled pan was recorded. A vacuum pen was used to transfer the sample pan from the weighing boat to the DSC sample holder. An empty reference pan was placed in the right sample holder. The sample housing was then closed and a thermogram was obtained.

Next the dry weight of the tissue samples was taken using a mass balance. The thermogram was then analyzed.

The water content of the tissue was obtained from DSC samples using the equation:

$$WaterContent = \frac{{Weight}_{dry}}{{Weight}_{wet} - {Weight}_{dry}}$$

### Results and Conclusions

### Data Analysis

Each thermogram was analyzed using the calculate peak area tool in the Pyris software to collect the onset temperature, peak temperature, and energy. The energy and dry tissue weight were then used to calculate the enthalpy of each sample. These calculations were performed in Excel.

### Statistical Analysis

To analyze the tissue a two-tailed student's t-test was used and a power analysis was preformed using GLM univariate. The results of the analysis are shown in Table 7.

TABLE 7
T-test and Power Analysis Results

| | | p-value | Power (%) |
|---|---|---|---|
| Leaflet | Onset Temp | 0.374 | 13.8 |
| | Peak Temp | 0.183 | 25.8 |
| | Enthalpy | 0.174 | 26.7 |
| Sinus | Onset Temp | 0.125 | 43.9 |
| | Peak Temp | 0.043 | 72.1 |
| | Enthalpy | 0.007 | 94.9 |
| Wall | Onset Temp | 0.003 | 90.6 |
| | Peak Temp | 0.412 | 12.4 |
| | Enthalpy | 0 | 100 |

### Results

Tables 8 and 9 below show the results from the DSC thermograms. The average onset temperature was within 1° C. between decellularized and cryopreserved tissue for leaflet, sinus and wall. The average peak temperature was also within 1° C. between decellularized and cryopreserved tissue for leaflet, sinus and wall. The average enthalpy had a wider range, from 2.378955 J/g to 17.94838 J/g. The samples shown to be statistically significant between cryopreserved and decellularized were the peak temperature and enthalpy of the sinus (p-values 0.043 and 0.007 respectively) as well as the onset temperature and the enthalpy of the wall (p-values 0.003 and 0.000 respectively).

TABLE 8
Decellularized Samples

| | Avg. | Std. Dev | | Avg. | Std. Dev. | | | |
|---|---|---|---|---|---|---|---|---|
| | Onset | Onset | | Peak | Peak | | Avg. | Std. Dev. |
| | Temp. | Temp. | | Temp. | Temp. | | Enthalpy | Enthalpy |
| | [C.] | [C.] | | [C.] | [C.] | | [J/g] | [J/g] |
| Leaflet | 64.2775 | 0.540654 | Leaflet | 66.1925 | 0.444771 | Leaflet | 17.94838 | 4.706242 |
| Sinus | 64.7775 | 0.63648 | Sinus | 67.36125 | 1.048733 | Sinus | 7.697944 | 2.817576 |
| Wall | 63.43429 | 0.449365 | Wall | 66.24714 | 0.194483 | Wall | 4.487269 | 0.690281 |

TABLE 9
Cryopreserved Samples

| | Avg. | Std. Dev | | Avg. | Std. Dev. | | | |
|---|---|---|---|---|---|---|---|---|
| | Onset | Onset | | Peak | Peak | | Avg. | Std. Dev. |
| | Temp. | Temp. | | Temp. | Temp. | | Enthalpv | Enthalpy |
| | [C.] | [C.] | | [C.] | [C.] | | [J/g] | [J/g] |
| Leaflet | 64.076 | 0.394721 | Leaflet | 65.882 | 0.489054 | Leaflet | 13.48328 | 7.795092 |
| Sinus | 64.382 | 0.138468 | Sinus | 66.442 | 0.228366 | Sinus | 4.015256 | 1.052852 |
| Wall | 64.038 | 0.261695 | Wall | 66.152 | 0.249212 | Wall | 2.378955 | 0.644986 |

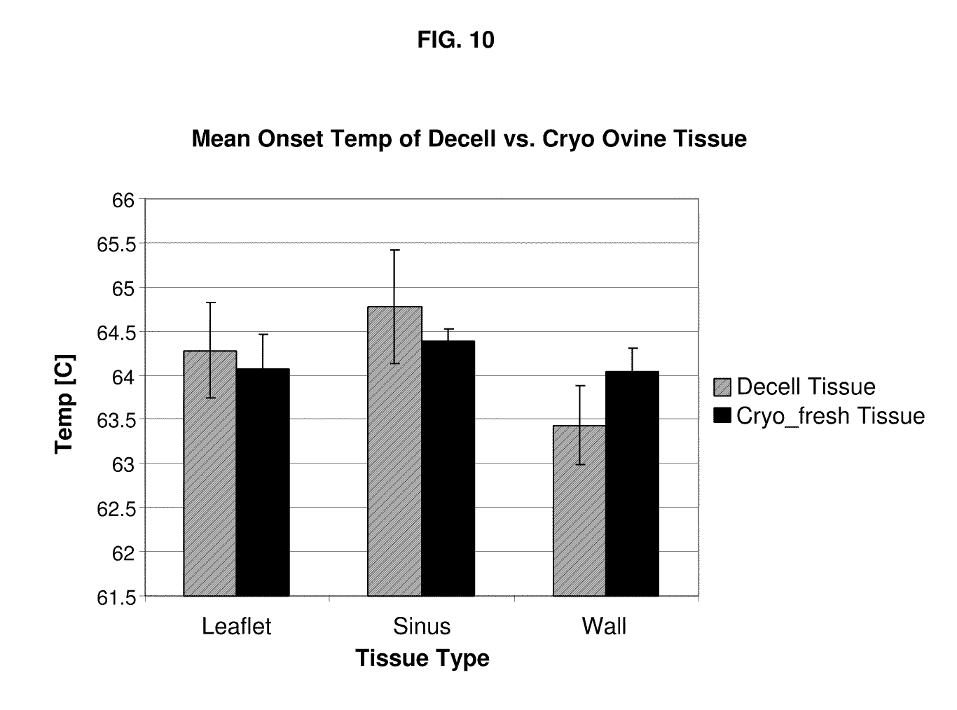

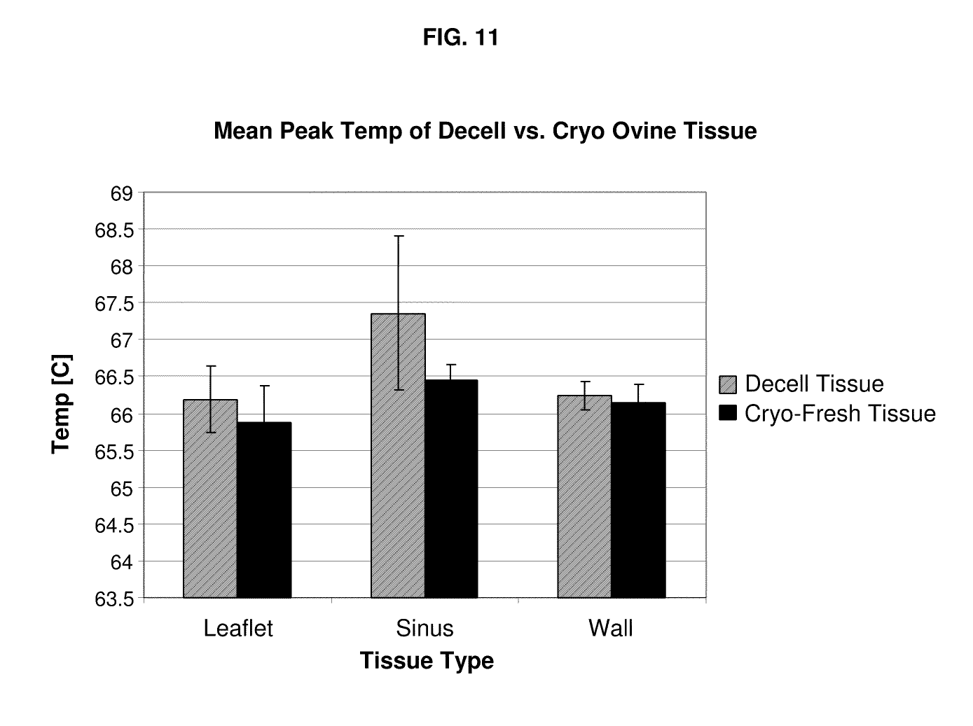

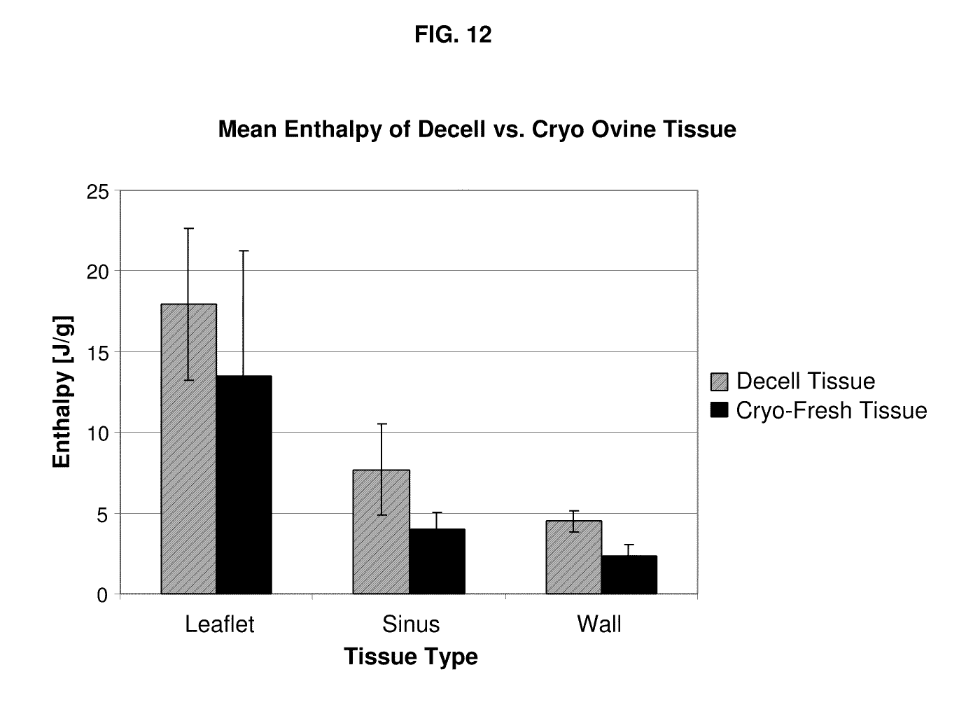

FIGS. 10, 11 and 12 are plots of the mean onset temperature, mean peak temperature and mean enthalpy between the decellularized and cryopreserved tissue samples.

One cusp was taken from two omega decellularized valves and one cusp was taken from two cryopreserved ovine pulmonary valves for this testing. Eight decellularized and ten cryopreserved tissue samples from wall, sinus and leaflet each were dissected and tested in the DSC.

This testing showed that there were significant differences between cryopreserved and decellularized tissues for the peak temperature and enthalpy of the sinus (p-values 0.043 and 0.007 respectively) as well as the onset temperature and the enthalpy of the wall (p-values 0.003 and 0.000 respectively).

Also, no significant differences were observed between cryopreserved and decellularized tissues for onset temperature, peak temperature and enthalpy for the leaflet (p-values 0.374, 0.183, and 0.174 respectively) onset temperature of the sinus (p-value 0.125) and peak temperature of the wall (p-value 0.412). The p-values for the data were compared to the significance threshold of 0.05.

The leaflet tissue showed no real difference between the cryopreserved and decellularized tissue for the variables tested. The sinus showed no difference in the onset temperature but a significant difference in the peak temperature and the enthalpy between the two tissue types. While the peak temperature of the wall showed no difference but the onset temperature and the enthalpy were significant. The increase in peak temperature and enthalpy in the decellularized sinus and vessel wall tissue was small (e.g. glutaraldehyde crosslinking would increase peak temperature by at least 30° C.) and thus, these data indicate no major changes in collagen crosslinking—either disruptive, (which would decrease the temperatures), or major increases in irreversible mature crosslinks that would prevent the collagen from being used by the restored cell population for structural protein synthesis—degradation cycle for adaptive and constructive remodeling.

### Example 8

This example illustrates the biomechanical properties of tissue decellularized according to the present invention.

### Materials and Methods

The materials and equipment used were as follows: Bose Planar Biaxial Test Bench, Calibrated Load Cell, Surgical scissors, Forceps, Weighing paper, Scalpel, Adhesive backed sandpaper, 0.9% Saline, Digital Calipers, and Flat-head screw driver.

Test and control articles were dissected into leaflet, sinus and wall regions. A single tissue strip (nominal width of 4 mm) was then dissected from each anatomic region (i.e., leaflet, sinus, wall). The actual dimensions of each strip were measured using digital calipers and recorded prior to tensile testing. The width of leaflet specimens was recorded, while the width and thickness of sinus and wall specimens were recorded. Specimens were loaded in uniaxial tension to failure using a crosshead speed of 10 mm/min. Specimens were pre-conditioned at 0.75 N and 1 Hz for 30 loading cycles prior to loading to failure. Load-deflection data was collected continuously throughout the tensile test. Testing was performed in 0.9% saline at 37° C. to simulate physiologic conditions.

### Data Analysis

Ultimate tensile strength (UTS) and elastic modulus (E) were calculated from load-deflection data. Independent samples t-tests were performed to determine statistically significant differences between experimental groups, with a level of significance of p=0.05.

### Results and Conclusions

Tensile test results are shown in tables 10-12. Decellularization resulted in significant increases in the ultimate UTS of leaflet (p=0.02) and sinus (p=0.04) tissue, and the elastic modulus of leaflet tissue (p=0.02). Other differences in the calculated material properties between experimental groups were not statistically significant (p<0.05).

TABLE 10
Ultimate tensile strength of cryopreserved and omega
decellularized ovine pulmonary valve tissues. Reported
values are in N/m for leaflet specimens and kPa for
sinus wall and pulmonary artery specimens.

| | Cryo | | | Decellularized | | |
|---|---|---|---|---|---|---|
| | | Sinus | Pulmonary | | Sinus | Pulmonary |
| | Leaflet | Wall | Artery | Leaflet | Wall | Artery |
| Mean | 1009.21 | 740.63 | 339.65 | 1352.23 | 1059.17 | 510.95 |
| Std. Dev. | 240.75 | 237.79 | 69.74 | 339.67 | 375.65 | 354.18 |

TABLE 11
Elastic modulus of cryopreserved and omega decellularized ovine
pulmonary valve tissues. Reported values are in N/m for leaflet
specimens and kPa for sinus wall and pulmonary artery specimens.

| | Cryo | | | Decellularized | | |
|---|---|---|---|---|---|---|
| | | Sinus | Pulmonary | | Sinus | Pulmonary |
| | Leaflet | Wall | Artery | Leaflet | Wall | Artery |
| Mean | 3648.24 | 2155.12 | 808.61 | 4559.77 | 2668.95 | 1252.29 |
| Std. Dev. | 649.62 | 775.38 | 206.34 | 898.64 | 978.26 | 765.11 |

Test and control articles were dissected into leaflet, sinus and wall regions and loaded in uniaxial tension to failure using a crosshead speed of 10 mm/min. Specimens were pre-conditioned at 0.75 N and 1 Hz for 30 loading cycles prior to loading to failure. Testing was performed in 0.9% saline at 37° C. to simulate physiologic conditions.

Ultimate tensile strength (UTS), strain-to-failure and elastic modulus (E) were calculated from load-deflection data. Independent samples t-tests were performed to determine statistically significant differences between experimental groups, with a level of significance of p=0.05.

Decellularization, in accordance with the present invention, resulted in a significant increase in the ultimate UTS of leaflet (p=0.02) and sinus (p=0.04) tissue, and the elastic modulus of leaflet tissue (p=0.02). Other differences in the calculated material properties between experimental groups were not statistically significant (p<0.05).

No significant difference was shown between the cryopreserved and decellularized leaflet samples for onset temperature, peak temperature or enthalpy. The sinus tissue only showed no difference in the onset temperature between the tissue types. However there was a significant difference in the peak temperature and the enthalpy for the sinus. The peak temperature of the wall showed no difference but the onset temperature and the enthalpy were significant. From these results there may be a difference between the collagen cross-linking for some of the areas of the sinus and arterial wall.

### Example 9

This example illustrates MHCI removal during the decell process of the present invention.

### Materials and Methods

The equipment and materials used were as follows: Mini-Protean Tetra Cell (Bio Rad #165-3301) or equivalent, Mini Trans-Blot Electrophoretic Transfer Cell (Bio Rad #170-3930) or equivalent, 10×TBS (Tris-Buffered Saline) pH 7.5 (Bio Rad #170-6435), 10× Tris/Glycine Buffer pH 8.3 (Bio Rad #161-0734), 10× Tris/Glycine/SDS Buffer pH 8.3 (Bio Rad #161-0732), Methanol, 2-mercaptoethanol (Bio Rad #161-0710) or equivalent, 10× Tris-Glycine SDS Sample Buffer (Invitrogen #LC2675), Tween 20 (Bio Rad #170-6531) or equivalent, Protein molecular weight marker of choice, Immobilon Western Chemiluminescent HRP detection kit (Millipore #WBKLS0500) or equivalent, Blotting membrane of choice (nitrocellulose, PVDF), X-ray film and film processor for autoradiography or imaging system, Hypercassette Autoradiography Cassettes (Amersham #RPN 11629), 0.25% Trypsin-EDTA (Invitrogen #25200-056), 4-15% Precast Gels (Bio Rad #161-1104) or equivalent, Surgical scissors or microtome blade, Forceps, Thermo Scientific Pierce BCA Protein Assay Kit (Fisher #23227), Fisher Scientific PowerGen Model 500 Homogenizer (Fisher #14-261-04), Thermo Scientific Pierce Prediluted BSA Protein Assay Standard Set (Fisher #23208), Blotting Grade Non-Fat Dry Milk (Bio Rad 170-6404), Bovine Serum Albumin (BSA) (Fisher # BP1605-100), 1×PBS (Invitrogen #10010-023), Ice, Ice Bucket, Dry ice, Micropipettors, Pipette tips, Gel Loading Tips, Primary antibody, Secondary antibody, species specific, Stripping buffer (Pierce #46430), Centrifuge, Centrifuge tubes (1.5 ml, 15 ml, 50 ml), Heating block, Refrigerator (4° C.), Freezer (−20° C., −80° C.), Whatman membrane marking pen (#10499001), Incubation trays (Falcon INTEGRID™ Petri Dish #351112), Plastic Sheets (cut up Ziploc bags or saran wrap), and Total Protein Extraction Kit (Millipore #2140).

On Day 1, fresh transfer buffer was created and stored. Next, electrophoresis buffer was created and stored. Next, 5% milk blocking buffer for routine blotting was prepared and stored. Wash buffer was then prepared along with the total protein extraction kit. Finally, 10× Tris-Glycine SDS Sample Buffer (10× Sample Buffer) was prepared. These solutions were prepared for use in a Western Blot Assay. Next, the samples were prepared for the Western Blot. The protein assay was completed, the gel was run and then the gel was transferred.

On Day 2, the primary antibody was prepared if not ready-to-use from the manufacturer. Finally, the HRP-conjugated secondary antibody was prepared if it is not ready-to-use from the manufacturer. The results of the Western Blot were analyzed for the presence of MHC I.

### Results and Conclusions

MHC I expression in test articles decellularized using the methods of the present invention was either not detectable or insignificant relative to its expression in native test articles. MHC I expression in decellularized test articles was either not detectable or insignificant relative to its expression in native test articles.

Current valve replacement options include stentless, glutaraldehyde fixed porcine valves and stented, glutaraldehyde fixed porcine leaflets. The stentless variety retains their native valve architecture of tissue leaflet and conduit components while the stented variety is composed of native leaflets sutured to a polymer conduit. Over time, the glutaraldehyde leaches out of the tissue components. This loss of cross linking will eventually expose foreign antigens to the host, leading to calcification of the valve and its ultimate failure. Patients receiving these valves will likely undergo multiple operations to replace these ill-fated valves throughout the course of their lives. In this example, the effectiveness of removing cellular material and antigenicity by way of the decellularization process of the present invention is investigated.

Ovine pulmonary valves either underwent treatment for decellularization, according to the methods of the present invention (Ω Decell) to remove cellular debris or were obtained native with cellular material intact after harvest. Test articles CSKC-09-130, CSKC-09-147, CSKC-10-3 and CSKC-10-4 were native and test articles CSKC-09-140, CSKC-09-141, CSKC-09-146 and CSKC-09-147 were decellularized according to the present invention. The difference between these groups of test articles becomes apparent when looking at MHC I expression. Lanes A3 and A4 of CSKC-09-147 show negligible expression when compared to lanes A5 and A6 of CSKC-09-141 and lane A2 (positive control; obtained from ovine cardiac muscle). The decellularization process clearly reduces the antigenicity of these implant candidates.

MHC I is expressed consistently in this test article in two different areas of the valve. MHC I levels are again negligible when compared to control. MHC I expression in both test articles is equal to that of control.

Two important conclusions can be drawn from the data contained herein. First, the present invention's decellularization process significantly reduces the level of MHC I expression in the valves tested for this study. Second, MHC I is a marker to predict the successful removal of cellular debris by the present invention's method. When taken together, these two findings suggest that S2 Decell processing can be used to prepare a Bioengineered Personal Heart Valve with a validated method to qualify each valve processed in this manner.

### Example 10

This example illustrates another embodiment of the present invention.

### Materials and Methods

### Reagent Preparation

A 0.05% Triton-X® (v/v) solution was prepared. First, a graduated cylinder was filled with deionized water and placed on a stir plate. A magnetic stir bar was then added. Next, the Triton-X® solution was added. Then, the stir plate was turned on and the speed was increased until the Triton-X® began to mix with the water. The solution was mixed until the Triton-X® was dissolved. Next, the solution was transferred to a beaker containing the remaining amount of deionzed water and placed on a stir plate with a magnetic stir bar. The two solutions were then combined and transferred to a beaked in a laminar flow hood. The solution was then stored at room temperature.

Next, a 1% (v/v) n-lauroyl sarcosine (NLS) solution was prepared. 100 mL n-lauroyl sarcosine was added to 1900 mL deionized water in a beaker. The solution was mixed using a stir plate and a magnetic stir bar. The solution was then stored at room temperature. Then, a Hypertonic Salt Solution (HSS) was prepared. NaCl, MgCl₂, mannitol and KCl were weighed out into weigh boats and transferred to a beaker. Saline was then added to bring the solution up to final volume. The solution was then mixed using stir plate and magnetic stir bar until salts were dissolved. The solution was then stored at room temperature. Final concentrations of reagents in HSS were 1.8% (w/v) NaCl, 12.5% (w/v) or ˜683 mM Mannitol, ˜2.3 mM M gCl₂, and 500 mM KCl in water.

A Saline Mannitol Solution (SMS) was then prepared. First, NaCl and mannitol were weighed out into weigh boats and transferred to a beaker. Then normal saline was added to bring the solution up to final volume. The solution was then mixed using stir plate and magnetic stir bar until salts are dissolved. The solution was then stored at room temperature.

Next, an Organic Solvent Extraction Buffer (ETOH) solution was prepared. Ethanol was measured out into a graduated cylinder and poured into a beaker. A magnetic stir bar was then added. Deionized water was added and the solution was mixed. The solution was then stored at room temperature. Final concentrations of reagents in SMS were 2.7 (w/v) NaCl and 12.5% or ˜683 mM Mannitol.

The following was the procedure used on each day of the investigation over a 4 day period of time:

On Day 1, 200 wide-mouth jars were filled with 167 mL of indicated solution using sterile serological pipette. All non-sterile items were then sprayed with 70% ethanol. Tissue was then removed from the cryo freezer storage. The tissue was then allowed tissue to sit at on the bench top for 7 minutes using a laboratory timer. The outer pouch was then opened and placed in the inner pouch into a basin. Up to 500 mL warm water (neither hot nor cold to the touch) was then added. Next, the inner pouch was allowed to sit in warm water for 7 minutes using a laboratory timer. The inner pouch was then transferred to a laminar flow hood. The inner pouch was then opened with sterile scissors and cryomedia was poured off. Using sterile forceps, tissue was transferred to a sterile bowl containing up to 200 mL Lacated Ringers' solution. The tissue was then allowed to sit in Lactated Ringer's solution for a minimum of 7 minutes using a laboratory timer. Using sterile forceps, tissue was transferred to a wide mouth jar labeled HSS. The jars were then transferred to a shaking incubator and set to 21° C. and 220 RPM. The jars were then incubated for 2 hours using laboratory timer. At the end of 2 hours, the tissue was transferred to a laminar flow hood. Using sterile forceps, tissue was transferred to a wide mouth jar labeled with Triton-X®. The jars were transferred to a shaking incubator and set to 21° C. and 220 RPM. The jars were incubated for 3 hr using laboratory timer. At the end of 3 hours, the tissue was transferred to a laminar flow hood. Using sterile forceps, the tissue was dipped into a wide mouth jar labeled with deionized water. Using sterile forceps, the tissue was transferred to a wide mouth jar labeled with deionized water. The jars were transferred to a shaking incubator and the incubator was set to 21° C. and 220 RPM. The tissue was then incubated for 10 min using a laboratory timer. At the end of 10 min, the tissue was transferred to a laminar flow hood. Using sterile forceps, the tissue was transferred to a wide mouth jar labeled with HSS. The jars were then transferred to a shaking incubator and set to 21° C. and 220 RPM. The jars were then incubated for 2 hr using a laboratory timer. At the end of 2 hours, the tissue was transferred to a laminar flow hood. Using sterile forceps, the tissue was transferred to a wide mouth jar labeled with deionized water. The jars were then transferred to a shaking incubator and set to 21° C. and 220 RPM. The tissue was then incubated for 1 hr using a laboratory timer. At the end of 1 hour, the tissue was transferred to a laminar flow hood. Using sterile forceps, the tissue was transferred to a wide mouth jar labeled with Triton-X®. The jars were transferred to a shaking incubator and set to 21° C. and 220 RPM. The jars were then incubated for 3 hours using a laboratory timer.

While the tissue was incubating, a Benzonase® solution was created. A beaker containing deionized water was obtained and a magnetic stir bar was placed inside and the beaker placed on a stir plate. Next, the Benzonase® vials were placed into a microcentrifuge and spun for several seconds. Using a 100 mL pipettor, the entire contents of Benzonase® vial was transferred into deionized water and allowed to mix. Next, the solution pH was measured with a pH meter. Using a 100 mL pipettor, NH₄OH was added until the pH of the solution reached 9-10. The solution was then stored at room temperature until use. Final concentrations of reagents in Benzonase® solution were 0.0625 KU/ml Benzonase® and 8 mM MgCl₂ in deionized water with a final pH after sterile filtration around 8.0.

At the end of the 3 hr Triton-X® incubation, the tissue was transferred to laminar flow hood. Using sterile forceps, the tissue was transferred to wide mouth jar labeled with Benzonase®. The jars were then transferred to a shaking incubator and set to 37° C. and 220 RPM. The jars were then incubated for 12 hr using laboratory timer.

On Day 2, the tissue was transferred to a laminar flow hood. Using sterile forceps, the tissue was transferred to a wide mouth jar labeled with deionized water. The jars were then transferred to a shaking incubator and set to 21° C. and 220 RPM. The tissue was then incubated for 1 hour using a laboratory timer. The tissue was then transferred to s laminar flow hood. Using sterile forceps, the tissue was transferred to wide mouth jar labeled with NLS. The jars were then transferred to a shaking incubator and set to 21° C. and 220 RPM. The tissue was then incubated for 24 hours using laboratory timer.

On Day 3, the tissue was transferred to a laminar flow hood. Using sterile forceps, the tissue was transferred to a wide mouth jar labeled with deionized water. The jars were then transferred to shaking incubator and set to 21° C. and 220 RPM. The tissue was then incubated for 2 hours using a laboratory timer. The tissue was then transferred to a laminar flow hood. Using sterile forceps, the tissue was transferred to a wide mouth jar labeled with ETOH. The jars were then transferred to a shaking incubator and set to 21° C. and 50 RPM. The tissue was then incubated for 30 min using a laboratory timer.

Next, the organic extraction bioreactor was prepared. Support frames were assembled around stir plates. The bioreactors were placed with u-traps and tubing under the laminar flow hood. The bioreactors (small and large) and tubing were then assembled. Then, the bioreactors were removed from the laminar flow hood. The large bioreactor was then placed on a left-hand stir plate. The small bioreactor was placed on a right-hand stir plate. The u-traps were then secured to the support frame. Tubing was then run through MiniPlus-3 and sterile 50 cc syringe was attached to the ports in the tubing to aid in water flow. Next, water was added to the bioreactors to a level just below the neck of the bioreactor. Then, the MiniPlus-3 was turned on and set to the highest setting. The settings were adjusted as needed to equilibrate flow. Water was circulating between the two bioreactors. Using sterile forceps the tissue was transferred to a basket in organic exchange bioreactor. The organic exchange resin beads (50 mL of each) were then added. Then, the stir plates were turned on to a setting where the stirrers moved freely within the bioreactors. The tissue was allowed to remain in the bioreactor for 24 hr.

On Day 4, the stir plates and water circulation were turned off. Using sterile forceps, the tissue was transferred to a wide mouth jar labeled with SMS. The jars were then transferred to a shaking incubator and set to 21° C. and 50 RPM. Then the tissue was incubated for 2 hours using a laboratory timer. At the end of 2 hours, tissue was cryopreserved

### Results and Conclusions

The decellularization process removes cell debris from the tissue and does so without a harmful effect on the heart valve. The tissue preparation allows for donor cells to easily infiltrate or be transferred and proliferate or be grown in the decellularized tissue. The decellularization process also prevents the calcification of the heart valve, leading to additional problems.

### Example 11

This example will evaluate ovine aortic valve leaflet visoelasticity.

### Materials and Methods:

Ovine aortic valve leaflet viscoelasticity will be evaluated using uniaxial and biaxial testing techniques. A total of 96 valves will be required to complete all aspects of the proposed studies.

Cryopreservation

Valves requiring cryopreservation will be initially cryopreserved within 72 hours of harvest using controlled rate freezers. Selected valves will then be subjected to mechanical testing following this initial cryopreservation procedure. Valves allocated for decellularization will be thawed, decellularized and subjected to a second cryopreservation procedure. Note that valves will remain in cryostorage for at least 24 hours following each cryopreservation procedure

Decellularization

Decellularization will be performed using methods in which ovine aortic heart valves are subjected to a reciprocating osmotic shock and multi-detergent and enzymatic washout protocol to remove cellular material.

Recellularization

Selected valves will be recellularized using a bioreactor based cell seeding protocol, utilizing autologous bone marrow from the recipient sheep under cyclic pressure loading conditions.

The passive effects of cellular material on aortic valve leaflet viscoelasticity will be determined. This will be accomplished by measuring the strain-rate dependence of leaflet tissue mechanical properties (i.e., storage modulus, loss modulus and hysteresis) in uniaxial and equi-biaxial stress states. Creep and stress-relaxation testing will also be performed in uniaxial and equi-biaxial stress states. Leaflet tissue will be dissected from fresh, cryopreserved and decellularized ovine aortic valves and cut into 4 mm wide strips for testing performed in uniaxial tension. Specimens of both circumferential and radial orientation will be tested. Uniaxial testing will be performed on an electromagnetic test instrument (Bose Biodynamic System, Bose Corp., Eden Prairie, Minn.) in Hank's Balanced Salt Solution at 37° C. To evaluate the strain-rate dependence of leaflet mechanical properties in uniaxial tension, specimens will be cyclically loaded to a maximum membrane tension of 60 N/m at frequencies of 0.5, 1 and 2 Hz. Leaflet specimens for creep and stress-relaxation testing will be loaded to a maximum membrane tension of 60 N/m using a rise time of 100 ms, followed by a 1 h hold period. Creep testing will performed under load control, while stress-relaxation testing will be performed in displacement control. All testing methods will be performed on specimens of both circumferential (n=9) and radial (n=9) orientation.

Aortic valve leaflet tissue designated for equi-biaxial testing will be dissected into 10×10 mm specimens. Biaxial testing will be performed on an electromagnetic test instrument (Bose Planar Biaxial System, Bose Corp., Eden Prairie, Minn.) in Hank's Balanced Salt Solution at 37° C. Specimens will be mounted to two pairs of opposing electromagnetic motors using 2.0 Prolene sutures. One “baseball stitch” suture will be used per specimen side to allow for a uniform application of load, with four evenly spaced attachment points per side. The circumferential and radial specimen directions will be aligned with the X and Y loading axis, respectively. Four small graphite marker dots will be adhered to test specimens to allow strain measurement via a video extensometer. To evaluate the strain-rate dependence of leaflet planar biaxial mechanical properties, specimens will be cyclically loaded to a maximum equi-biaxial membrane tension of 60 N/m at frequencies of 0.5, 1 and 2 Hz. Leaflet specimens for creep and stress-relaxation testing will be loaded to a maximum membrane tension of 60 N/m using a rise time of 100 ms, followed by a 1 h hold period. Creep testing will performed under load control, while stress-relaxation testing will be performed in displacement control.

In addition to mechanical testing, morphology and biochemical assays will be performed to evaluate the structure and composition of native, cryopreserved and decellularized specimens. Histology and DNA quantification (98+% removal of native DNA=decellularized) will be performed to verify decellularization and to observe structural proteins and ground substances in the extracellular matrix. Transmission electron microscopy will be used to observe leaflet ultrastructure. Biochemical assays will be performed to quantify collagen, elastin, glycosaminoglycan, and total protein content of leaflet specimens.

The effects of cellular contraction on aortic valve leaflet viscoelasticity will be determined. This will be accomplished by measuring the stress-relaxation behavior of aortic valve leaflet tissue subjected to pharmacological treatments designed to induce cellular contraction. Ovine aortic valves harvested will be shipped overnight in hypothermosol at 4° C. to maintain cell viability. Specimens will be tested within 36 hours of harvest. Equi-biaxial stress relaxation testing will be performed in oxygenated Kreb's buffer at 37° C. The pH, as well as the O₂ and CO₂ content, of the test media will be maintained at constant levels throughout the course of the experiment. To assess the viscoelastic properties of aortic valve leaflet tissue in the absence of cellular contraction (passive properties), a vasodilative agent (e.g., 5-hydroxytrypatamine) will be added to the test media during mechanical testing. Similarly, to assess the properties of the tissue in the presence of cellular contraction (active properties), a vasoconstrictive agent (e.g., endothelin-1) will be added to the test media. Aortic valve leaflet specimens will be subjected to an equi-biaxial membrane tension of 60 N/m in displacement control and allowed to relax for a period of 10 minutes. Specimens will then be unloaded and allowed to relax for 10 minutes, followed by a second stress-relaxation experiment performed using an increased concentration of the vasoconstrictive or vasodilative agent in the test media. Load-deflection data will be collected continuously throughout the duration of the experiment.

The effects of recellularization on aortic valve leaflet viscoelasticity will be determined. This will be accomplished by measuring the stress-relaxation behavior of aortic valve leaflet tissue following recellularization. Ovine aortic valves will be decellularized and subsequently recellularized using a bioreactor based cell seeding strategy, utilizing autologous bone marrow from the recipient sheep under cyclic pressure loading conditions. The recellularized valves will then be implanted for 20 weeks in sheep to further mature the reestablish the cell population. The recellularized aortic valves will be implanted in the pulmonary valve position to increase animal survivability (aortic valve replacement in sheep is technically difficult due to a deep V-shaped thorax and short ascending aorta). Cryopreserved and decellularized ovine aortic valves will also be implanted. Selected donor valves will be implanted with pulmonary artery banding distal to the implanted valve so as to pressure load to the level of systemic pressures. All of these chronic surgical models are successfully and regularly performed in our laboratory. Two leaflets from each explanted valve will be allocated for biaxial stress-relaxation testing. Thus, the experimental groups comprise cryopreserved aortic valves with (CW, n=4) or without (CWO, n=4) pulmonary artery banding decellularized aortic valves with (DW, n=6) and without (DWO, n=6) pulmonary artery banding and recellularized aortic valves with (RW, n=6) or without (RWO, n=6) pulmonary artery banding. Testing will be performed in oxygenated Kreb's buffer with and without a vasoconstrictor or vasodilator at 37° C. The pH, as well as the O₂ and CO₂ content, of the test media will be maintained at constant levels throughout the course of the experiment. Specimens will be subjected to an equi-biaxial membrane tension of 60 N/m in displacement control and allowed to relax for a period of 1 hour. Selected recellularized valves from either bioreactor in vitro cell seeding (under cyclic pressure loading) using autologous donor cells (eg, bone marrow; vascular smooth muscle cells; other appropriate cell types) or in vivo autologous recellularized cusps obtained from decellularized valves implanted in the right ventricular outflow tract in sheep.

This study will be performed as an addition to a scheduled chronic implant study. The scheduled study will comprise the implantation of 3 cryopreserved, ovine aortic valves, 4 decellularized, ovine aortic valves and 4 recellularized, ovine aortic valves in the pulmonary position.

Data Analysis:

A number of material properties will be measured during mechanical testing. The storage modulus, loss modulus and hysteresis of tissue samples will be calculated from load-deflection data to determine the strain-rate dependence of leaflet mechanical properties under uniaxial and biaxial loading conditions. Additionally, peak stretches in the circumferential and radial specimen directions will be recorded during biaxial testing. Load-deflection data will be collected continuously throughout the time course of creep and stress relaxation experiments. Creep will be reported in terms of the change in stretch in the circumferential at multiple time points throughout the experiment. Stress-relaxation will be reported in terms of the percent relaxation at the conclusion of the experiment. Mean valves for the measured and calculated parameters will be determined for the native, decellularized and conditioned experimental groups. Independent samples t-tests will be used to test for significant differences between experimental groups. Statistical analyses will be performed using SPSS version 17.0 software (SPSS Inc., Chicago, Ill.), with a level of significance of p=0.05.

Groups comprising 9 specimens will be used for all mechanical testing as described herein.

### Results and Conclusions

The results will show that the decellularized valves perform better and possess better mechanical properties than valves decellularized by methods other than those of the present invention or that are cryopreserved after such other methods.

### Example 12

This example illustrates a further DSC study performed on heart valves decellularized according to the methods of the present invention.

### Materials and Methods

This study used a DSC to test leaflet, sinus, and arterial wall tissue of native cryopreserved and omega decellularized ovine pulmonary tissues. The DSC examines collagen cross-linking and denaturation temperature of the samples by analysis of heat flow changes during a ramped heating protocol. Data for peak denaturation temperature, onset temperature, and enthalpy was collected for each sample. The data from this study aided in determining how the decellularization process affects the valve tissue.

### Test Articles

| | | | | Secondary |
|---|---|---|---|---|
| Accession | Spe- | | Valve | Accession |
| numbers | cies | Tissue Type | Type | numbers |
| CSKC-09-123 | Ovine | Ω Decellularized | Pulmonary | 9DV |
| CSKC-09-125 | Ovine | Ω Decellularized | Pulmonary | 4DV |

### Control Articles

| | | | | Secondary |
|---|---|---|---|---|
| Accession | Spe- | | Valve | Accession |
| numbers | cies | Tissue Type | Type | numbers |
| CSKC-09-130 | Ovine | Cryopreserved | Pulmonary | 14DV |
| | | Native | | |
| CSKC-09-141 | Ovine | Cryopreserved | Pulmonary | 7-DV |
| | | Native | | |

### Sample Preparation

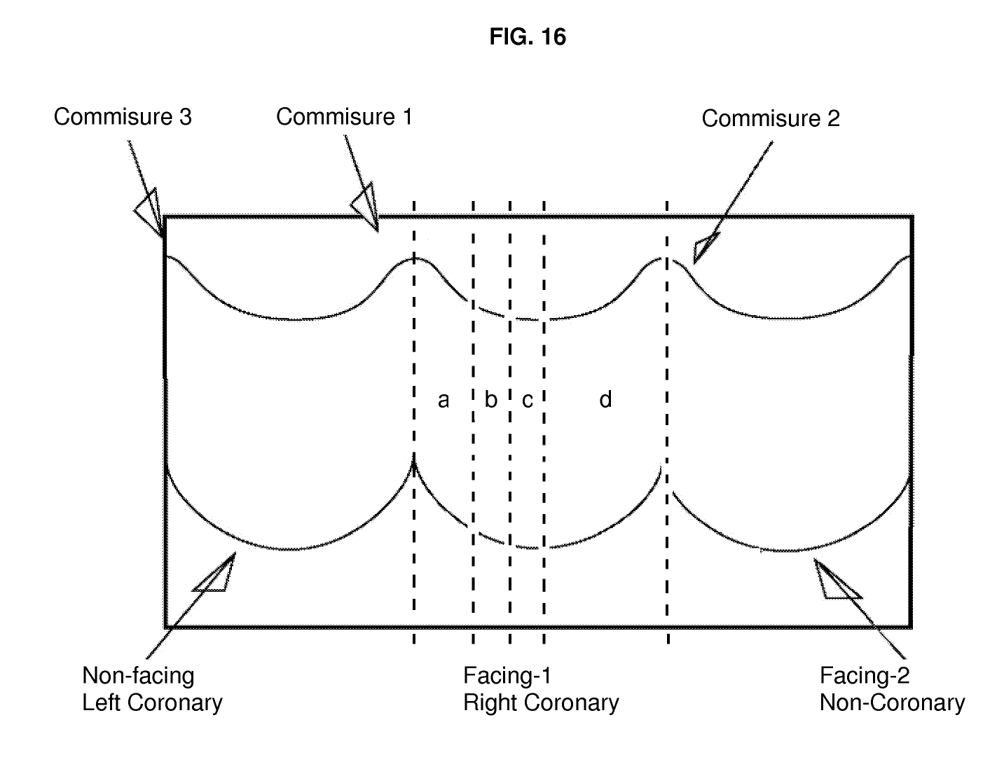

Two decellularized and two cryopreserved valves were dissected into 3 cusps according to FIG. 16. Using a predetermined valve allocation matrix one of the 3 dissected cusps for each valve was prepared for DSC testing.

The cusp for DSC was further dissected into leaflet, sinus, and arterial wall. Eight tissue specimens from the decellularized tissue and ten from the cryopreserved tissue were cut out of each leaflet, sinus, and arterial wall. Using a mass balance, their mass was recorded as a wet tissue weight. The dissected tissue samples were approximately 5 mg samples wet weight. They were placed in aluminum sample pans, sealed, weighed again and taken to the DSC for testing. These specimens were then tested in the DSC by heating the tissue in a ramped protocol from 40° C. to 90° C. by 5° C./min and thermograms were generated for each sample. From the thermograms the onset temperature, peak temperature and enthalpy were collected. The pans were punctured and placed in an oven overnight to dry the tissue. The dry pan weight was measured and recorded so the moisture content of the tissue could be calculated as well as the dry tissue mass.

### Data Analysis

Each thermogram was analyzed using the “calculate peak area” tool in the Pyris software to collect the onset temperature, peak temperature, and energy. The energy (the area under the peak on the thermogram) and dry tissue weight were then used to calculate the enthalpy of each sample. Enthalpy is the amount of heat transfer to a mass and in our case it is the amount of heat absorbed by the tissue. The Enthalpy equation is listed below.

$$Enthalpy = \frac{Area of Thermogram Peak [ j ]}{Mass of Dry Tissue [ g ]}$$

### Statistical Analysis

To analyze the tissue a two-tailed student's t-test with N=2 was used with a significance level of 0.05.

### Results and Conclusions

Table 12 below shows the results from the calculations of DSC thermograms. The mean onset temperature and mean peak temperatures were all within 1° C. between decellularized and cryopreserved tissue for leaflet, sinus and wall. The average enthalpy had a slightly wider range, with variations up to 4.46 J/g for the leaflet samples. These plots include the mean peak temperature, mean onset temperature and enthalpy as well as the standard deviation of each.

TABLE 12
Results of the thermogram analysis are as follows:

| | | Avg. | | Avg. | | | |
|---|---|---|---|---|---|---|---|
| | | Onset | Std. Dev | Peak | Std. Dev. | Avg. | Std. Dev. |
| | | Temp. | Onset | Temp. | Peak | Enthalpy | Enthalpy |
| | | [C.] | Temp. [C] | [C.] | Temp. [C.] | [J/g] | [J/g] |
| Decellularized | Leaflet | 64.2775 | 0.540654 | 66.1925 | 0.444771 | 17.94838 | 4.706242 |
| | Sinus | 64.7775 | 0.63648 | 67.36125 | 1.048733 | 7.697944 | 2.817576 |
| | Wall | 63.43429 | 0.449365 | 66.24714 | 0.194483 | 4.487269 | 0.690281 |
| Cryopreserved | Leaflet | 64.076 | 0.394721 | 65.882 | 0.489054 | 13.48328 | 7.795092 |
| | Sinus | 64.382 | 0.138468 | 66.442 | 0.228366 | 4.015256 | 1.052852 |
| | Wall | 64.038 | 0.261695 | 66.152 | 0.249212 | 2.378955 | 0.644986 |

A student's T-test was used to look for significant differences between the decellularized and cryopreserved tissues. There was no statistical difference between the decellularized and cryopreserved samples for any of the groups at a significance level of 0.05. This data suggests that there are no significant differences in the collagen cross-linking of the decellularized tissue.

TABLE 13
Differences between decellularized tissue portions
when compared to cryopreserved tissue portions

| | | p-Value |
|---|---|---|
| Leaflet | Onset Temp | 0.487 |
| | Peak Temp | 0.449 |
| | Enthalpy | 0.42 |
| Sinus | Onset Temp | 0.417 |
| | Peak Temp | 0.481 |
| | Enthalpy | 0.332 |
| Wall | Onset Temp | 0.214 |
| | Peak Temp | 0.666 |
| | Enthalpy | 0.417 |

One cusp was taken from two omega decellularized valves and one cusp was taken from two cryopreserved ovine pulmonary valves for this testing. A total of eight decellularized and ten cryopreserved tissue samples from wall, sinus and leaflet each were dissected and tested in the DSC. The DSC heated the samples from 40° C. to 90° C. by increments of 5° C./min. DSC thermograms were collected and data for the peak temperature, onset temperature and enthalpy were collected and calculated from the thermograms. The onset temperature for all the samples was around 64.5° C. for both cryopreserved and decellularized tissue. Also the peak temperature was fairly consistent for all the samples with values around 66.2° C. for both cryopreserved and decellularized tissue.

This testing showed that there were no significant differences between cryopreserved and decellularized tissues for the peak temperature, onset temperature or enthalpy of the leaflet, sinus, or wall. The p-values, shown in the table below, were all much greater than 0.05 with the smallest being 0.214 for the onset temperature of the wall.

TABLE 14
P-values for the onset temperature of cryopreserved
when compared to decellularized tissues

| | | p-Value |
|---|---|---|
| Leaflet | Onset Temp | 0.487 |
| | Peak Temp | 0.449 |
| | Enthalpy | 0.42 |
| Sinus | Onset Temp | 0.417 |
| | Peak Temp | 0.481 |
| | Enthalpy | 0.332 |
| Wall | Onset Temp | 0.214 |
| | Peak Temp | 0.666 |
| | Enthalpy | 0.417 |

These results suggest that the current omega decellularization process used, does not significantly (p-value 0.05) alter the collagen cross-linking of pulmonary valve tissue. The data also indicates that this is true for all three regions of a pulmonary valve: the leaflet, sinus, and wall.

### Example 13

This example illustrates another investigation into the amount of MHC I expression

### Materials and Methods

The decellularization process was designed to reduce or eliminate cellular debris and therefore any traces of antigenicity from candidate implantable biological scaffolds. Every nucleated cell expresses a molecule on its surface to facilitate the detection of proteins normally found in the body, called the Major Histocompatibility Complex I (MHC I). For this reason, MHC I was chosen as a marker for detecting cellular debris.

The following tissue samples and test article accession numbers were used in this study:

### Test Articles

| | | | | Secondary |
|---|---|---|---|---|
| Accession | Spe- | | Valve | Accession |
| Numbers | cies | Tissue Type | Type | Numbers |
| CSKC-09-140 | Ovine | Ω Decellularized | Pulmonary | 6DV |
| CSKC-09-141 | Ovine | Ω Decellularized | Pulmonary | 7DV |
| CSKC-09-146 | Ovine | Ω Decellularized | Pulmonary | 4333 |
| CSKC-09-148 | Ovine | Ω Decellularized | Pulmonary | 4313 |
| CSKC-09-130 | Ovine | Cryopreserved | Pulmonary | 14DV |
| | | Native | | |
| CSKC-09-147 | Ovine | Cryopreserved | Pulmonary | P5068 |
| | | Native | | |
| CSKC-10-3 | Ovine | Cryopreserved | Pulmonary | 13DV |
| | | Native | | |
| CSKC-10-4 | Ovine | Cryopreserved | Pulmonary | 16DV |
| | | Native | | |

### Control Articles

### Sample Preparation

Four decellularized and four cryopreserved valves were dissected into 3 cusps according to FIG. 16. Using a predetermined valve allocation matrix, one of the 3 dissected cusps from each valve was prepared for Western Blotting.

The cusp for Western Blotting was not further dissected into leaflet, sinus and arterial wall as further dissection made decellularized samples too dilute to include in the assay. Each whole cusp was minced into ˜1 mm² pieces and was placed in lysis buffer for 1 hour on ice. The minced tissue was then homogenized with a saw-toothed generator and was placed on ice for 15 minutes. After the 15 minute incubation on ice, the homogenates were clarified by centrifugation at 21,000×g for 30 minutes at 4° C. The clarified homogenate was removed from the tissue debris and was frozen at −80° C. until the Western Blot assay could be performed. The samples were run on a 4-20% Tris-Glycine gradient gel (Invitrogen, # EC6025BOX) for ˜90 minutes at 150 V.

The samples were transferred to nitrocellulose membranes overnight at 35 V in a 4° C. refrigerator. The next morning, the nitrocellulose membrane was blocked for 1 hour at room temperature in 5% Milk/TBS-T (0.3% Tween-20) blocking buffer. The membrane was probed with MHC I antibody (Santa Cruz Biotechnology, # sc-59205) for 1 hour at room temperature with gentle agitation.

### Data Analysis

Data analysis was done in a qualitative fashion. The relative expression of MHC I was visually compared in decellularized test articles and their native control counterparts.

### Results and Conclusions

MHC I expression in decellularized test articles was either not detectable or insignificant relative to its expression in native test articles.

The table below summarizes the Western Blot results:

TABLE 15

| Accession | | |
|---|---|---|
| Numbers | Tissue Type | MHC I |
| CSKC-09-130 | Cryopreserved Native | + |
| CSKC-09-140 | Ω Decellularized | ↓ |
| CSKC-09-141 | Cryopreserved Native | ++ |
| CSKC-09-146 | Ω Decellularized | ↓ |
| CSKC-09-147 | Ω Decellularized | ↓ |
| CSKC-09-148 | Ω Decellularized | ↓ |
| CSKC-10-3 | Cryopreserved Native | ++ |
| CSKC-10-4 | Cryopreserved Native | ++ |

+ = normal expression; ++ = high expression; ↓ = low expression.

In this report, the effectiveness of removing cellular material and antigenicity by the decellularization process was investigated, as measured by the presence of MHC I.

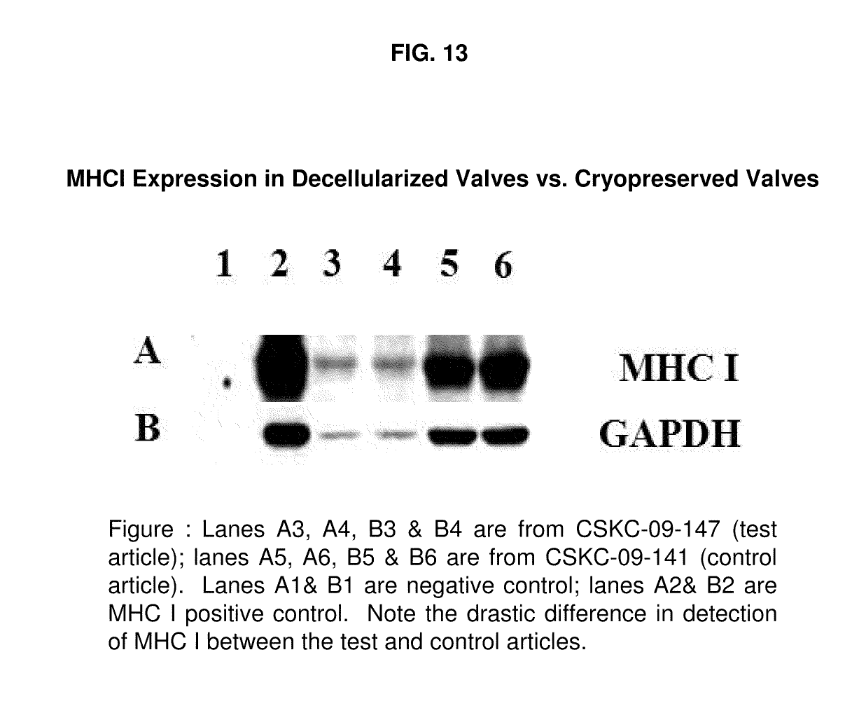

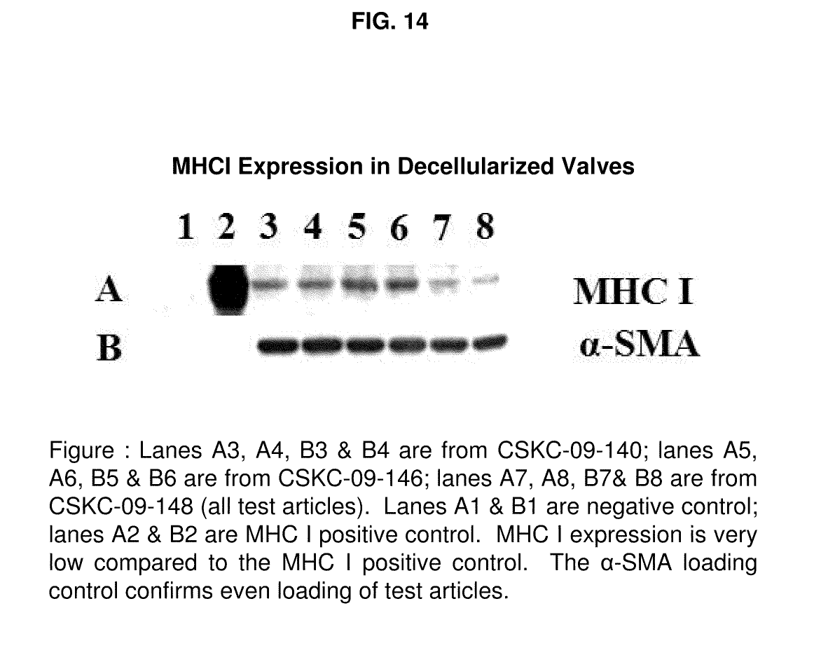

Ovine pulmonary valves either underwent treatment for decellularization to remove cellular debris or were obtained native with cellular material intact after harvest. Test articles CSKC-09-130, CSKC-09-147, CSKC-10-3 and CSKC-10-4 were native and control articles CSKC-09-140, CSKC-09-141, CSKC-09-146 and CSKC-09-147 were decellularized. The difference between these groups of test articles became apparent when looking at MHC I expression. Lanes A3 and A4 of CSKC-09-147 show negligible expression when compared to lanes A5 and A6 of CSKC-09-141 and lane A2 (positive control; obtained from ovine cardiac muscle), as shown in FIG. 13. The decellularization process clearly reduces the antigenicity of these implant candidates. FIG. 14 illustrates that MHC I expression is very low compared to the MHC I positive control. In FIG. 14, lanes A3-A8 are all decellularized valves.

MHC I is expressed consistently in this test article in two different areas of the valve. MHC I levels are again negligible when compared to control. MHC I expression in both test articles is equal to that of control.

The results contained herein show that the Ω decellularization process is effective in removing cellular material from the biological scaffolds tested. MHC I was never completely removed from the decellularized test articles, however the significant reduction in its detection in test articles as compared to control articles is proof of its ability to reduce cellular material and therefore antigenicity from an implantable bioengineered personal heart valve.

What is claimed is:

1. A method for removing cells from a tissue comprising the steps of:
a. obtaining a harvested tissue:
b. performing a muscle shelf debridement on said tissue;
c. contacting said tissue with an enzyme;
d. contacting said tissue with a detergent; and
e. performing an organic solvent extraction on said tissue.

2. The method of claim 1, wherein said tissue is selected from the group consisting of: heart tissue, lung tissue, liver tissue, pancreas tissue, small intestine tissue, large intestine tissue, colon tissue, spleen tissue, and gland tissue.

3. The method of claim 1, wherein said method is completed over the course of 2-14 days.

4. The method of claim 1, wherein said detergent is selected from the group consisting of a nonionic detergent, anionic detergent, zwitterionic detergent, and combinations thereof.

5. The method of claim 4, wherein a nonionic detergent is used first followed by the use of an anionic or zwitterionic detergent.

6. The method of claim 1, wherein said enzyme is an endonuclease.

7. The method of claim 1, wherein said organic solvent extraction uses an alcohol.

8. The method of claim 1, wherein said organic solvent extraction uses a salt.

9. The method of claim 1, wherein said organic solvent extraction uses a sugar alcohol.

10. The method of claim 1, wherein said muscle shelf debridement comprises an enzymatic debridement by which dead, contaminated, or adherent tissue or foreign materials are removed from said tissue.

11. A method for removing cells from a tissue comprising the steps of:
a. obtaining a harvested tissue;
b. performing at least one reciprocating osmotic shock sequence on said tissue;
c. contacting said tissue with a detergent;
d. performing an RNA-DNA extraction on said tissue;
e. contacting said tissue with an enzyme; and
f. performing an organic solvent extraction on said tissue.

12. The method of claim 11, wherein said tissue is selected from the group consisting of: heart tissue, lung tissue, liver tissue, pancreas tissue, small intestine tissue, large intestine tissue, colon tissue, spleen tissue, and gland tissue.

13. The method of claim 11, wherein said method is completed over the course of 2-14 days.

14. The method of claim 11, wherein said detergent is selected from the group consisting of a nonionic detergent, anionic detergent, zwitterionic detergent, and combinations thereof.

15. The method of claim 14, wherein a nonionic detergent is used first followed by the use of an anionic or zwitterionic detergent.

16. The method of claim 11, wherein said enzyme is an endonuclease.

17. The method of claim 11, wherein said organic solvent extraction uses an alcohol.

18. The method of claim 11, wherein said organic solvent extraction uses a salt.

19. The method of claim 11, wherein said organic solvent extraction uses a sugar alcohol.

20. The method of claim 11, wherein said osmotic shock sequences use a Hypertonic Salt Solution.

21. The method of claim 20, wherein said Hypertonic Salt Solution uses a sugar alcohol.

22. The method of claim 11, wherein said RNA-DNA extraction uses an enzyme.

23. The method of claim 11, wherein said RNA-DNA extraction uses MgCl.

24. The method of claim 11, further comprising the step of contacting said tissue with ddH₂O.

25. A method for removing cells from a tissue comprising the steps of:
a. obtaining a harvested tissue;
b. performing a first reciprocating osmotic shock sequence on said tissue;
c. contacting said tissue with a first detergent;
d. performing a second reciprocating osmotic shock sequence on said tissue;
e. performing a RNA-DNA extraction on said tissue;
f. performing a digestion on said tissue;
g. contacting said tissue with an enzyme;
h. contacting said tissue with a second detergent;
i. performing a first organic solvent extraction on said tissue;
j. performing an ion-exchange detergent residual extraction on said tissue; and
k. performing a second organic solvent extraction on said tissue.

26. A method for removing the cells from a tissue comprising the steps of:
a. a first contacting of said tissue with a Hypertonic Salt Solution;
b. a first contacting of said tissue with Triton X®;
c. a first contacting of said tissue with ddH₂O;
d. a second contacting of said tissue with a Hypertonic Salt Solution;
e. a second contacting of said tissue with ddH₂O;
f. a second contacting of said tissue with Triton X®;
g. performing a Benzonase® digestion on said tissue;
h. a third contacting of said tissue with ddH₂O;
i. contacting said tissue with a 1% NLS solution;
j. a fourth contacting of said tissue with ddH₂O;
k. contacting said tissue with 40% EtOH;
l. performing an organic solvent extraction on said tissue; and
m. contacting said tissue with SMS.

27. A decellularized tissue prepared using at least 5 of the following steps:
a. obtaining a harvested tissue:
b. performing a muscle shelf debridement on said tissue;
c. contacting said tissue with an enzyme;
d. performing at least one reciprocating osmotic shock sequence on said tissue;
e. performing an RNA-DNA extraction on said tissue;
f. contacting said tissue with a first detergent;
g. performing a digestion on said tissue;
h. contacting said tissue with a second detergent;
i. performing a first organic solvent extraction on said tissue;
j. performing an ion-exchange detergent residual extraction on said tissue;
k. performing a second organic solvent extraction on said tissue
l. a first contacting of said tissue with a Hypertonic Salt Solution;
m. a first contacting of said tissue with Triton X®;
n. contacting said tissue with a Hypotonic Salt Solution;
o. a first contacting of said tissue with ddH₂O;
p. a second contacting of said tissue with a Hypertonic Salt Solution;
q. a second contacting of said tissue with ddH₂O;
r. a second contacting of said tissue with Triton X®;
s. performing an endonuclease digestion on said tissue;
t. a third contacting of said tissue with ddH₂O;
u. contacting said tissue with a 1% NLS solution;
v. a fourth contacting of said tissue with ddH₂O;
w. contacting said tissue with 40% EtOH;
x. performing an organic solvent extraction on said tissue; and
y. contacting said tissue with SMS.

28. The tissue of claim 27, wherein said tissue has a characteristic selected from the group consisting of: containing little or no dsDNA, exhibiting less calcification after implant into an animal when compared to tissues prepared not using at least 5 steps of claim 27, having a reduced inflammatory response when compared to tissues prepared not using at least 5 steps of claim 27, having an increased ultimate tensile strength when compared to tissues prepared not using at least 5 steps of claim 27, having an increased elastic modulus when compared to tissues prepared not using at least 5 steps of claim 27, and combinations thereof.
